(19) United States
(12) Patent Application Publication
(10) Pub. No.: US 2009/0264648 A1
(43) Pub. Date: Oct. 22, 2009

(54) SYNTHESIS OF PYRAZOLES

(75) Inventors: Warren Chew, Pierrefonds (CA); Zheng Wang, East Brunswick, NJ (US); Gloria Cheal, Beaconsfield (CA); Martial Bertrand, Laval (FR); John Potoski, New Marlborough, MA (US); Mahmoud Mirmehrabi, Laval (CA); Arianna Nencini, Castelnuovo Berardenga (IT); Riccardo Zanaletti, Colle Val d'Elsa (IT)

Correspondence Address: CHOATE, HALL & STEWART LLP, TWO INTERNATIONAL PLACE, BOSTON, MA, 02110, US

(73) Assignees: WYETH, Madison, NJ (US); SIENA BIOTECH S.P.A., Siena (IT)

(21) Appl. No.: 12/353,896

(22) Filed: Jan. 14, 2009

Related U.S. Application Data
(60) Provisional application No. 61/021,015, filed on Jan. 14, 2008.

(51) Int. Cl. C07D 243/08

(52) U.S. Cl. 540/575

(57) ABSTRACT

The present invention provides compounds and compositions, methods of making them, and methods of using them to modulate α7 nicotinic acetylcholine receptors and/or to treat any of a variety of disorders, diseases, and conditions. Provided compounds can affect, among other things, neurological, psychiatric and/or inflammatory system.

### CROSS REFERENCE TO RELATED APPLICATIONS

This application claims priority to U.S. provisional patent application Ser. No. 61/021,015, filed Jan. 14, 2008, the entirety of which is hereby incorporated herein by reference.

### FIELD OF THE INVENTION

The present invention relates to the synthesis of compounds with α7 nicotinic acetylcholine receptor (α7 nAChR) agonistic activity, derivatives thereof, and intermediates thereto.

### BACKGROUND OF THE INVENTION

Agents that bind to nicotinic acetylcholine receptors have been indicated as useful in the treatment and/or prophylaxis of various diseases and conditions, particularly psychotic diseases, neurodegenerative diseases involving a dysfunction of the cholinergic system, and conditions of memory and/or cognition impairment, including for example, schizophrenia, anxiety, mania, depression, manic depression, Tourette's syndrome, Parkinson's disease, Huntington's disease, cognitive disorders (such as Alzheimer's disease, Lewy Body Dementia, Amyotrophic Lateral Sclerosis, memory impairment, memory loss, cognition deficit, attention deficit, Attention Deficit Hyperactivity Disorder), and other uses such as treatment of nicotine addiction, inducing smoking cessation, treating pain (e.g. analgesic use), providing neuroprotection, and treating jetlag. See for example WO 97/30998; WO 99/03850; WO 00/42044; WO 01/36417; Holladay et al., J. Med. Chem., 40:26, 4169-94 (1997); Schmitt et al., Annual Reports Med. Chem., Chapter 5, 41-51 (2000); Stevens et al., Psychopharmatology, (1998) 136: 320-27; and Shytle et al., Molecular Psychiatry, (2002), 7, pp. 525-535.

Different heterocyclic compounds carrying a basic nitrogen and exhibiting nicotinic and muscarinic acetylcholine receptor affinity or claimed for use in Alzheimer's disease have been described, e.g. 1H-pyrazole and pyrrole-azabicyclic compounds (WO2004013137); nicotinic acetylcholine agonists (WO2004039366); ureido-pyrazole derivatives (WO0112188); oxadiazole derivatives having acetylcholinesterase-inhibitory activity and muscarinic agonist activity (WO9313083); pyrazole-3-carboxylic acid amide derivatives as pharmaceutical compounds (WO2006077428); arylpiperidines (WO2004006924); ureidoalkylpiperidines (U.S. Pat. No. 6,605,623); compounds with activity on muscarinic receptors (WO9950247). In addition, modulators of alpha7 nicotinic acetylcholine receptor are disclosed in WO06008133, in the name of the same applicant.

### SUMMARY

Among other things, the invention provides methods of preparing compounds acting as full or partial agonists at the α7 nicotinic acetylcholine receptor (α7 nAChR), and intermediates thereof. Such compounds are useful for the treatment of diseases that may benefit from the activation of the alpha 7 nicotinic acetylcholine receptor such as neurological, neurodegenerative, psychiatric, cognitive, immunological, inflammatory, metabolic, addiction, nociceptive, and sexual disorders, in particular Alzheimer's disease, schizophrenia, and/or others. Such compounds include those of formula I-1:

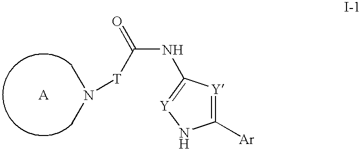

or a pharmaceutically acceptable salt thereof, wherein each of Ar, Y, Y′, T, and Ring A is as defined herein.

The present invention also provides synthetic intermediates useful for preparing such compounds. The invention further provides methods to provide cost effective yields and purity.

### BRIEF DESCRIPTION OF THE DRAWING

FIGS. 1-7 show characterization data for hydrochloride salts.

FIG. 8 illustrates the effect of pH and HCl equivalence on HCl salt formation.

FIG. 9 shows the effects of pH and HCl equivalence on HCl salt formation

FIGS. 10 and 11 depict conversion of higher HCl salts to mono-HCl forms.

FIG. 12 shows a DSC scan of 5-(4-acetyl-1,4-diazepan-1-yl)-N-(5-(4-methoxyphenyl)-1H-pyrazol-3-yl)pentanamide hydrochloric salt Form I.

FIG. 13 shows a TGA thermogram of 5-(4-acetyl-1,4-diazepan-1-yl)-N-(5-(4-methoxyphenyl)-1H-pyrazol-3-yl)pentanamide hydrochloric salt Form I.

FIGS. 14a-b show X-ray diffraction pattern and data for 5-(4-acetyl-1,4-diazepan-1-yl)-N-(5-(4-methoxyphenyl)-1H-pyrazol-3-yl)pentanamide hydrochloric salt Form I.

FIG. 15 presents DVS isothermal analysis of 5-(4-acetyl-1,4-diazepan-1-yl)-N-(5-(4-methoxyphenyl)-1H-pyrazol-3-yl)pentanamide hydrochloric salt Form I.

FIG. 16 is a DSC scan of 5-(4-acetyl-1,4-diazepan-1-yl)-N-(5-(4-methoxyphenyl)-1H-pyrazol-3-yl)pentanamide hydrochloric salt Form II.

FIG. 17 is a TGA thermogram of 5-(4-acetyl-1,4-diazepan-1-yl)-N-(5-(4-methoxyphenyl)-1H-pyrazol-3-yl)pentanamide hydrochloric salt Form II.

FIGS. 18a-b show X-ray diffraction pattern and data for 5-(4-acetyl-1,4-diazepan-1-yl)-N-(5-(4-methoxyphenyl)-1H-pyrazol-3-yl)pentanamide hydrochloric salt Form II.

FIG. 19 presents a DVS isothermal analysis of 5-(4-acetyl-1,4-diazepan-1-yl)-N-(5-(4-methoxyphenyl)-1H-pyrazol-3-yl)pentanamide hydrochloric salt Form II.

### DESCRIPTION OF CERTAIN PARTICULAR EMBODIMENTS

### Definitions

The term “aliphatic” or “aliphatic group,” as used herein, means a straight-chain (i.e., unbranched) or branched, hydrocarbon chain that is completely saturated or that contains one or more units of unsaturation, or a monocyclic hydrocarbon that is completely saturated or that contains one or more units of unsaturation, but which is not aromatic (also referred to herein as “carbocycle” “cycloaliphatic” or “cycloalkyl”), that has a single point of attachment to the rest of the molecule. In certain embodiments, aliphatic groups contain 1-6 aliphatic carbon atoms, and in yet other embodiments, aliphatic groups contain 1-3 aliphatic carbon atoms. In some embodiments, “cycloaliphatic” (or “carbocycle”) refers to a monocyclic C3-C6 hydrocarbon that is completely saturated or that contains one or more units of unsaturation, but which is not aromatic, that has a single point of attachment to the rest of the molecule. Such cycloaliphatic groups include cycloalkyl and cycloalkenyl groups. Suitable aliphatic groups include, but are not limited to, linear or branched alkyl, alkenyl, alkynyl groups and hybrids thereof such as (cycloalkyl)alkyl, (cycloalkenyl)alkyl or (cycloalkyl)alkenyl.

The term “lower alkyl,” as used herein, refers to a hydrocarbon chain having up to 6 carbon atoms, preferably 1 to 3 carbon atoms, and more preferably 1 to 2 carbon atoms. The term “alkyl” includes, but is not limited to, straight and branched chains such as methyl, ethyl, n-propyl, isopropyl, n-butyl, isobutyl, sec-butyl, or t-butyl.

The term “alkoxy,” as used herein, refers to the group —OR*, wherein R* is a lower alkyl group.

The term “acyl,” as used herein, refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{XI})OR^{XI}, —C(═NR^{XI})SR^{X1}, and —C(═NR^{X1})N(R^{X1})₂, wherein R^{XI} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{XI} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The terms “halogen” or “halo,” as used herein, refer to chlorine, bromine, fluorine or iodine.

The term “alkenyl,” as used herein refers to an aliphatic straight or branched hydrocarbon chain having 2 to 4 carbon atoms that has one or more double bonds. Examples of alkenyl groups include vinyl, prop-1-enyl, allyl, methallyl, but-1-enyl, but-2-enyl, or but-3-enyl. The term “lower alkenyl” refers to an alkenyl group having 1 to 3 carbon atoms.

The term “aryl,” as used herein refers to phenyl or an 8-10 membered bicyclic partially unsaturated or aryl ring. Exemplary aryl groups include phenyl and naphthyl. In certain embodiments, the term “aryl,” as used herein refers to an 8-10 membered bicyclic partially unsaturated the wherein at least one of the rings is aromatic.

The term “heteroaryl,” as used herein, refers to a 5-6 membered monocyclic heteroaryl having 1-4 heteroatoms independently selected from nitrogen, oxygen, or sulfur, or an 8-10 membered bicyclic partially unsaturated or heteroaryl ring having 1-5 heteroatoms independently selected from nitrogen, oxygen, or sulfur. Examples of heteroaryls include, but are not limited to, thienyl, furyl, pyrrolyl, imidazolyl, pyrazolyl, oxazolyl, isoxazolyl, thiazolyl, isothiazolyl, pyridyl, pyrazinyl, pyrimidinyl, pyridazinyl, indolyl, indazolyl, benzofuranyl, isobenzofuranyl, benzothienyl, isobenzothienyl, quinolyl, isoquinolyl, quinoxalinyl, or quinazolinyl.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{}, -(haloR^{}), —(CH₂)₀₋₂OH, —(CH₂)₀₋₂OR^{}, —(CH₂)₀₋₂CH(OR^{})₂; —O(haloR^{}), —CN, —N₃, —(CH₂)₀₋₂C(O)R^{}, —(CH₂)₀₋₂C(O)OH, —(CH₂)₀₋₂C(O)OR^{}, —(CH₂)₀₋₂SR^{}, —(CH₂)₀₋₂SH, —(CH₂)₀₋₂NH₂, —(CH₂)₀₋₂NHR^{}, —(CH₂)₀₋₂NR^{}₂, —NO₂, —SiR^{}₃, —OSiR^{}₃, —C(O)SR^{}, —(C₁₋₄ straight or branched alkylene)C(O)OR^{}, or —SSR^{} wherein each R^{} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —(C(R*₂))₂₋₃O—, or S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{}, -(haloR^{}), —OH, —OR^{Ø}, —O(haloR^{}), —CN, —C(O)OH, —C(O)OR^{}, —NH₂, —NHR^{}, —NR^{}₂, or —NO₂, wherein each R^{} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{}, -(haloR^{}), —OH, —OR^{}, —O(haloR^{}), —CN, —C(O)OH, —C(O)OR^{}, —NH₂, —NHR^{}, —NR^{}₂, or —NO₂, wherein each R^{} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “pharmaceutically acceptable salts” or “pharmaceutically acceptable salt” includes acid addition salts, that is salts derived from treating compounds of formulae I-1 and A with an organic or inorganic acid such as, for example, acetic, lactic, citric, cinnamic, tartaric, succinic, fumaric, maleic, malonic, mandelic, malic, oxalic, propionic, hydrochloric, hydrobromic, phosphoric, nitric, sulfuric, glycolic, pyruvic, methanesulfonic, ethanesulfonic, toluenesulfonic, salicylic, benzoic, or similarly known acceptable acids. Where a compound of formulae I-1 and A contains a substituent with acidic properties, for instance, phenolic hydroxyl, —SO₂H or —CO₂H, the term also includes salts derived from bases, for example, sodium salts.

In certain embodiments, the present invention provides a method for preparing compounds of formula I-1:

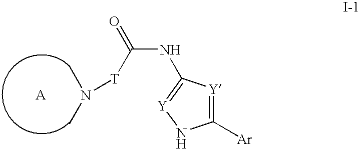

or a pharmaceutically acceptable salt thereof, wherein:
Ring A is a 4-8 membered saturated ring, having 0-2 heteroatoms independently selected from O, N, or S in addition to the nitrogen depicted in Ring A, wherein Ring A is independently substituted with 0-4 R′ groups
R′ is selected from the group consisting of mono- or di-[linear, branched or cyclic C₁₋₆ alkyl]aminocarbonyl; linear, branched or cyclic C₁₋₆ alkyl, alkoxy, or acyl;
Y and Y′ are each independently N or C, with the proviso that at least one of Y or Y′ is N;
T is a C₃₋₅ bivalent hydrocarbon chain, optionally carrying an oxo group and optionally substituted with one or more halogen, hydroxy, C₁₋₅ alkyl, alkoxy, fluoroalkyl, hydroxyalkyl, alkylidene, or fluoroalkylidene groups; C₃₋₆ cycloalkane-1,1-diyl, oxacycloalkane-1,1-diyl, C₃₋₆ cycloalkane-1,2-diyl, or oxacycloalkane-1,2-diyl groups, wherein the bonds of the 1,2-diyl radical form a fused ring with the T chain; and with the proviso that when T carries an oxo group, said oxo group is not part of an amide bond; and
Ar is a group selected from 6-10 membered aryl, or 5-10 membered heteroaryl having 1-4 heteroatoms independently selected from nitrogen, oxygen or sulfur; wherein Ar is optionally substituted with one or more substituents independently selected from halogen; hydroxy; mercapto; cyano; nitro; amino; sulfonyl; linear, branched or cyclic (C1-C6) alkyl, trihaloalkyl, di- or trihaloalkoxy, alkoxy, or alkylcarbonyl; (C3-C6) cycloalkyl-(C1-C6) alkoxy; (C3-C6) cycloalkyl-(C1-C6) alkyl; linear, branched, or cyclic (C1-C6) alkylcarbonylamino; mono- or di-, linear, branched, or cyclic (C1-C6) alkylaminocarbonyl; carbamoyl; linear, branched, or cyclic (C1-C6) alkylsulphonylamino; linear, branched, or cyclic (C1-C6) alkylsulphonyl; mono- or di-, linear, branched, or cyclic (C1-C6) alkylsulphamoyl; linear, branched or cyclic (C1-C6) alkoxy-(C1-C6) alkyl; wherein, two substituents may be taken together with their intervening atoms to form a ring.

In certain embodiments, the present compounds are generally prepared according to Scheme 1 set forth below:

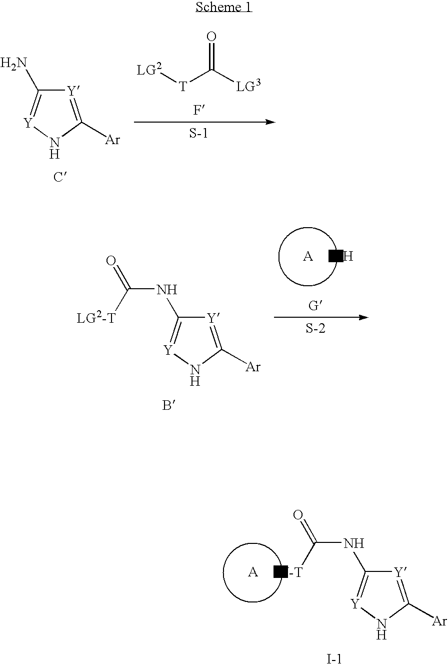

wherein each Ring A, Y, Y′, T, and Ar is defined in formula I-1 and described in classes and subclasses above and herein, and LG² and LG³ are leaving groups.

In certain embodiments, the T moiety on formulae I-1, B′, and F′ is a C₃₋₅ bivalent hydrocarbon chain, optionally substituted with one or more halogen, hydroxy, C₁₋₅ alkyl, alkoxy, fluoroalkyl, hydroxyalkyl, alkylidene, or fluoroalkylidene groups. In some embodiments, the T moiety is a C₄ bilvalent hydrocarbon chain.

In certain embodiments, Y is a nitrogen atom. In certain embodiments, Y′ is a nitrogen atom.

In certain embodiments, Ring A of a compound of formula G′ is a 4-8 membered saturated ring. In certain embodiments, a compound of formula G′ is a piperazine derivative. In certain embodiments, a compound of formula G′ is N-acetylhomopiperazine.

In certain embodiments, Ar is an optionally substituted group selected from 6-10 membered aryl, or 5-10 membered heteroaryl having 1-4 heteroatoms independently selected from nitrogen, oxygen or sulfur. In some embodiments, Ar is an optionally substituted 6 membered aryl group. In certain embodiments, Ar is methoxy-phenyl. In certain embodiments, Ar is 4-methoxy-phenyl. In some embodiments, Ar is hydroxy-phenyl. In some embodiments, Ar is a phenyl group substituted with a —OSO₃H group.

At step S-1, a compound of formula C′ is reacted in a suitable solvent, as defined and described herein, with a compound of formula F′ to form a compound of formula B′.

The LG² group of formulae B′ and F′ is a suitable leaving group, as defined and described herein. In certain embodiments, the LG² group of formulae B′ and F′ is halogen, —OMs, —OTs, or —OTf. In certain embodiments, the LG² group of formulae B′ and F′ is —Br.

The LG³ group of formula F′ is a suitable leaving group, as defined and described herein. In certain embodiments, the LG³ group of formula F′ is halogen, —OR,

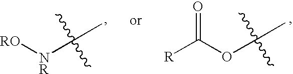

wherein each R is independently hydrogen or an optionally substituted group selected from C₁₋₆ aliphatic, 6-10 membered aryl, or 5-10 membered heteroaryl having 1-4 heteroatoms independently selected from nitrogen, oxygen or sulfur. Examples of LG³ groups of formula F′ include —OH, —OMe, —OEt, —Cl, —Br,

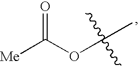

and the like. In some embodiments, LG³ is —Cl.

In certain embodiments, LG³ is —OH, and the reaction between a compound of formula F′ and a compound of formula B′ is carried out using suitable peptide coupling conditions. Suitable peptide coupling conditions are well known in the art and include those described in detail in Han et al., Tetrahedron, 60, 2447-67 (2004), the entirety of which is hereby incorporated by reference. In certain embodiments, the peptide coupling conditions include the addition of HOBt, DMAP, BOP, HBTU, HATU, BOMI, DCC, EDC, IBCF, or a combination thereof.

In certain embodiments, the compound of formula F′ is selected from 5-bromovaleryl chloride or 5-iodovaleryl chloride. In some embodiments, the compound of formula F′ is 5-bromovaleryl chloride.

One of ordinary skill in the art will appreciate that a variety of suitable leaving groups LG³ can be used to facilitate the reaction described in step S-1, and all such suitable leaving groups are contemplated by the present invention.

Suitable solvents for step S-1 include aprotic solvents, aliphatic halides, substituted and unsubstituted aromatic hydrocarbons, aliphatic ethers or combinations thereof. In certain embodiments, the solvent is selected from diethyl ether, t-butyl methyl ether, THF, ethyl acetate, DMSO, DMF, NMP, acetonitrile, dichloromethane, benzene, toluene, or combination thereof. In some embodiments, the solvent is a mixture of acetonitrile and DMF. In certain embodiments, the solvent is a 9:1 mixture of acetonitrile:DMF.

Suitable bases for step S-1 include tertiary amines such as pyridine, N-methylmorpholine, 1,4-diazabicyclo[2.2.2]octane, triethylamine (TEA), 1,8-diazabicyclo[5.4.0]undec-7-ene, diisopropylethylamine, and tetramethylethylenediamine; potassium carbonate, sodium bicarbonate, sodium carbonate, potassium hydroxide, sodium hydroxide, tetrabutylammonium hydroxide, benzyltrimethylammonium hydroxide, triethylbenzylammonium hydroxide, 1,1,3,3-tetramethylguanidine, and combinations thereof. In certain embodiments, the base is diisopropylethylamine.

Suitable temperatures at which the reaction described in step S-1 may occur include about −20° C. to about 60° C. In certain embodiments, the temperature is about −10° C. to about 25° C. In some embodiments, the temperature is about −10° C.

While not wishing to be bound by any particular theory, it is believed that the order of reagent addition may be useful in reducing the formation of byproducts in step S-1. In certain embodiments a compound of formula F′ is added slowly to a compound of formula C′.

It will be appreciated that certain reaction conditions may result in the formation of a regioisomer in step S-1 (i.e., reaction of endocyclic pyrazole nitrogen with a compound of formula F′). In certain embodiments, the product of step S-1 is treated to remove the undesired regioisomer. In some embodiments, the product of step S-1 is re-slurried with a particular solvent or solvents to remove the regioisomer. In some embodiments, the product of step S-1 is re-slurried with t-butyl methyl ether to remove the regioisomer.

At step S-2, a compound of formula B′ is reacted in a suitable solvent with a compound of formula G′, optionally in the presence of a suitable base and/or cataylst, to produce compound I-1. One of ordinary skill in the art will recognize that when a compound of formula B′ is 5-bromo-N-(5-(4-methoxyphenyl)-1H-pyrazol-3-yl)pentanamide, 5-iodo-N-(5-(4-methoxyphenyl)-1H-pyrazol-3-yl)pentanamide may be generated in situ by the addition of an iodide source, affording exchange of an iodine atom with the bromine atom. Examples of such iodide sources include, but are not limited to, sodium iodide, potassium iodide, hydrogen iodide, tetralkylammonium iodides, or mixtures thereof. While not wishing to be bound by any particular theory, it is believed that 5-iodo-N-(5-(4-methoxyphenyl)-1H-pyrazol-3-yl)pentanamide may be a more reactive species in step S-2 than 5-bromo-N-(5-(4-methoxyphenyl)-1H-pyrazol-3-yl)pentanamide, due to the greater leaving group character of iodide over bromide.

Suitable solvents for step S-2 include aprotic solvents, aliphatic halides, aliphatic ethers or combinations thereof. In certain embodiments, the solvent is selected from dichloromethane, diethyl ether, acetonitrile, THF, NMP, N-methyl morpholine, dimethylacetaminde, acetone, or combination thereof. In some embodiments, the solvent is acetone.

Suitable bases for step S-2 include tertiary amines such as pyridine, N-methylmorpholine, 1,4-diazabicyclo[2.2.2]octane, triethylamine (TEA), 1,8-diazabicyclo[5.4.0]undec-7-ene, diisopropylethylamine, and tetramethylethylenediamine; potassium carbonate, sodium bicarbonate, sodium carbonate, potassium hydroxide, sodium hydroxide, tetrabutylammonium hydroxide, benzyltrimethylammonium hydroxide, triethylbenzylammonium hydroxide, 1,1,3,3-tetramethylguanidine, and combinations thereof. In certain embodiments, the base is potassium carbonate. In certain embodiments, the base is diisopropylethylamine.

In certain embodiments, the iodide source used in step S-2 is sodium iodide. In certain embodiments, the iodide source is potassium iodide. In some embodiments, the iodide source is used in catalytic amounts ranging from about 0.1 to about 0.5 equivalents relative to a compound of formula B′. In other embodiments, the iodide source is used stoichiometrically relative to a compound of formula B′.

Suitable temperatures at which the reaction described in step S-2 may occur include about −20° C. to about 60° C. In certain embodiments, the temperature is about −10° C. to about 25° C. In some embodiments, the temperature is about 25° C.

One of ordinary skill in the art will recognize that a compound of formula I-1 may be further transformed into a pharmaceutically acceptable salt. In certain embodiments, the pharmaceutically acceptable salt is a hydrochloride salt. In some embodiments, the pharmaceutically acceptable salt is a mono hydrochloride salt.

In certain embodiments, as described in Example 5, an extractive workup step may be performed following step S-2. In certain embodiments, the organic phase comprises one or more organic solvents. In some embodiments, the organic solvents are ethanol, methyl tetrahydrofuran, dichloromethane, or a combination thereof. In certain embodiments, the solvents are ethanol and methyl tetrahydrofuran. In certain embodiments, the solvents are ethanol and dichloromethane. In some embodiments, the organic solvents are 5% ethanol in methyl tetrahydrofuran. In some embodiments, the organic solvents are 5% ethanol in dichloromethane.

In certain embodiments, dimer compounds may be formed as side products at step S-2. In such cases, it has been found that performing additional washes following HCl salt formation may be useful for reducing the presence of such dimer products.

In certain embodiments, each of the aforementioned synthetic steps may be performed sequentially with isolation of each intermediate B′ and I-1 performed after each step. Alternatively, each of steps S-1 and S-2, as depicted in Scheme I above, as well as any subsequent salt formation, may be performed in a manner whereby no isolation of one or more intermediates B′ and I-1 is performed. In certain embodiments, steps S-1 and S-2 are performed in sequence without any isolation of intermediates. In certain embodiments, step S-2 and salt formation are performed without any isolation of intermediates. While not wishing to be bound by any particular theory, it is believed that such techniques may be useful in obtaining compounds of formulae B′, I-1, and pharmaceutically acceptable salts thereof in greater yield and purity.

In certain embodiments, the present invention provides a method for preparing compound 1,5-(4-acetyl-1,4-diazepan-1-yl)-N-(5-(4-methoxyphenyl)-1H-pyrazol-3-yl)pentanamide hydrogen chloride:

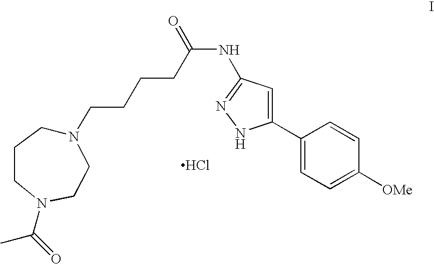

which may also be referred to as 5-(4-Acetyl-[1,4]diazepan-1-yl)-pentanoic acid [5-(4-methoxy-phenyl)-1H-pyrazol-3-yl]-amide.

Compound I, a potent α7 nAChR agonist, is effective in treating diseases that may benefit from the activation of the alpha 7 nicotinic acetylcholine receptor such as neurological, neurodegenerative, psychiatric, cognitive, immunological, inflammatory, metabolic, addiction, nociceptive, and sexual disorders, in particular Alzheimer's disease, schizophrenia, and/or others.

Certain methods of preparing compounds of the present invention are known in the art and include those described in detail in WO2006/008133 and U.S. Ser. No. 60/880,629, the entirety of each of which is hereby incorporated herein by reference.

In certain embodiments, the present compounds are generally prepared according to Scheme 2 set forth below:

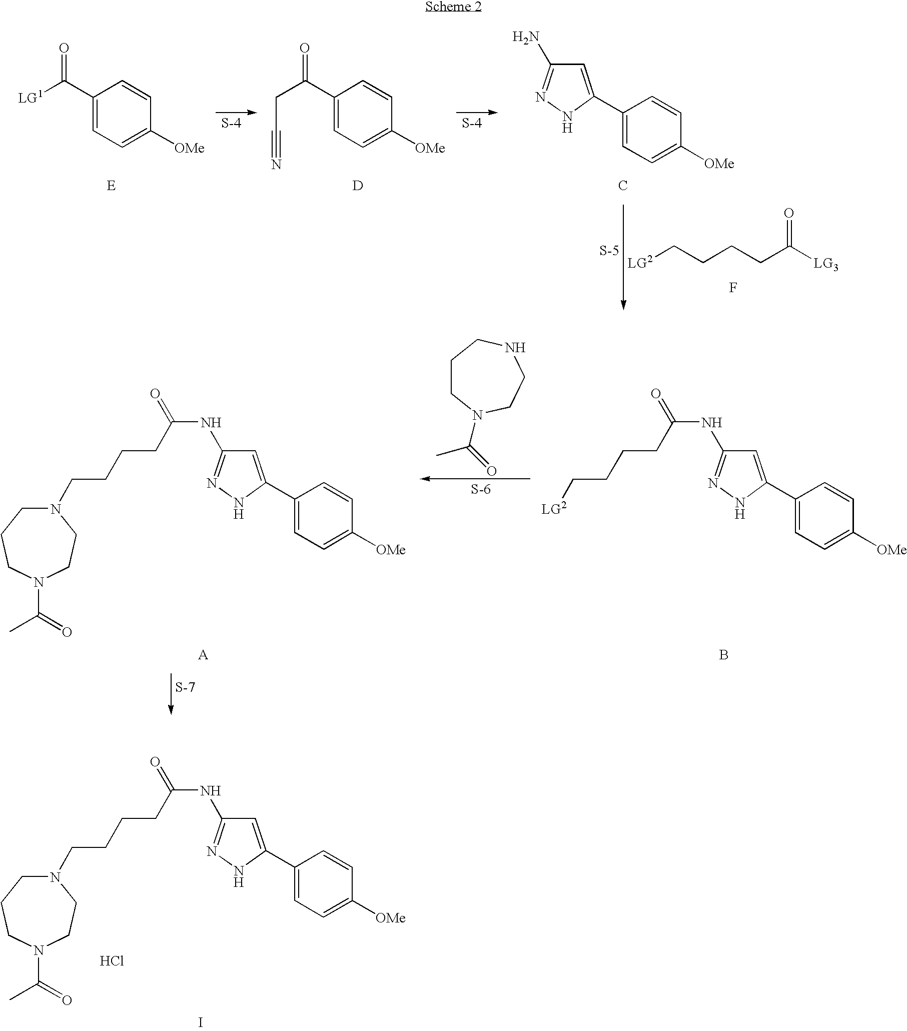

In one aspect, the present invention provides methods for preparing a free base, A, according to the steps depicted in Scheme 2, above. At step S-3, an aromatic ester of formula E is reacted, optionally in a suitable solvent, with a suitable base and acetonitrile to provide β-ketonitrile D. Such suitable bases include metal alkoxides and metal hydrides. In certain embodiments, the base is an alkali hydride, a metal alkyl, a metal amide, or a metal silazide. Other suitable bases include KH, n-butyl lithium, hexyl lithium, lithium diisopropylamide, Li—N(Si-alkyl)₂, lithium hexamethyldisilazide (LiHMDS), sodium hexamethyldisilazide (NaHMDS), potassium hexamethyldisilazide (KHMDS) potassium t-amylate, sodium t-butoxide (NaOtBu), and sodium hydride (NaH). In certain embodiments, the base is LiHMDS.

The acetonitrile used in step S-3 may be used in a range of equivalents relative to aromatic ester E. In certain embodiments, the equivalents of acetonitrile range from 0.5 to 50 equivalents. In certain embodiments, the equivalents of acetonitrile range from 2 to 10 equivalents. In some embodiments, the equivalents of acetonitrile range from 4 to 6 equivalents.

Suitable temperatures at which the reaction described in step S-3 may occur include about −20° C. to about 80° C. In certain embodiments, the temperature is about −20° C. to about 0° C. In some embodiments, the temperature is about −10° C.

Step S-3 may optionally employ a suitable solvent. A suitable solvent is a solvent or a solvent mixture that, in combination with the combined reacting partners and reagents, facilitates the progress and/or rate of the reaction. The suitable solvent may solubilize one or more of the reaction components, or, alternatively, the suitable solvent may facilitate the suspension of one or more of the reaction components; see, generally, “Advanced Organic Chemistry,” Jerry March, 5^{th} edition, John Wiley and Sons, N.Y.

Examples of solvents suitable for use in step S-3 include anhydrous aprotic solvents, such as aliphatic halides, substituted and unsubstituted aromatic hydrocarbons, aliphatic nitriles, and aliphatic ethers. In some embodiments, the solvent is selected from toluene, acetonitrile, diethyl ether, t-butyl methyl ether, THF, benzene, dichloromethane or combinations thereof. In certain embodiments, no solvent is used.

The LG¹ group of formula E is a suitable leaving group. A suitable leaving group is a chemical group that is readily displaced by a desired incoming chemical moiety. Suitable leaving groups are well known in the art, e.g., see, “Advanced Organic Chemistry,” Jerry March, 5^{th} Ed., pp. 351-357, John Wiley and Sons, N.Y. Such leaving groups include, but are not limited to, halogen, alkoxy, sulphonyloxy, optionally substituted alkylsulphonyl, optionally substituted alkenylsulfonyl, optionally substituted arylsulfonyl, and diazonium moieties. Examples of some suitable leaving groups include chloro, iodo, bromo, fluoro, methanesulfonyl (mesyl), tosyl, triflate, nitro-phenylsulfonyl (nosyl), and bromo-phenylsulfonyl (brosyl).

In certain embodiments, the LG¹ group of formula E is halogen, —OR,

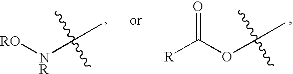

wherein each R is independently hydrogen or an optionally substituted group selected from C₁₋₆ aliphatic, 6-10 membered aryl, or 5-10 membered heteroaryl having 1-4 heteroatoms independently selected from nitrogen, oxygen or sulfur. Examples of LG¹ groups of formula E include —OH, —OMe, —OEt, Cl, Br,

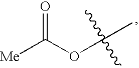

and the like. In some embodiments, LG¹ is —OR, wherein R is C₁₋₆ alkyl. In certain embodiments, LG¹ is —OR, wherein R is methyl.

One of ordinary skill in the art will appreciate that a variety of suitable leaving groups can be used to facilitate the reaction described in step S-3, and all such suitable leaving groups are contemplated by the present invention.

According to an alternate embodiment, the suitable leaving group may be generated in situ within the reaction medium. For example, a leaving group may be generated in situ from a precursor of that compound wherein said precursor contains a group readily replaced by said leaving group in situ.

At step S-4, β-ketonitrile D is reacted in a suitable solvent with hydrazine, or an equivalent thereof, to form aryl aminopyrazole C. Such hydrazine equivalents are well known to one of ordinary skill in the art and include, but are not limited to, anhydrates, hydrates, monohydrates, monohydrochlorides, dihydrochlorides, and sulfates. In certain embodiments, the hydrazine equivalent used in step S-4 is a hydrate. In some embodiments, the hydrazine equivalent is a monohydrate.

Suitable solvents for step S-4 include alkyl alcohols, such as C₁ to C₄ alcohols (e.g. ethanol, methanol, 2-propanol), aliphatic halides, substituted and unsubstituted aromatic hydrocarbons, or aliphatic ethers or combinations thereof. In certain embodiments, the solvent is selected from ethanol, toluene, dichloromethane, diethyl ether, THF, benzene or combination thereof. In some embodiments, the solvent is ethanol.

Suitable temperatures at which the reaction described in step S-4 may occur include about 10° C. to about 150° C. In certain embodiments, the temperature is about 30° C. to about 70° C. In some embodiments, the temperature is about 60° C.

At step S-5, aryl aminopyrazole C is reacted in a suitable solvent with a compound of formula F to form a compound of formula B.

The LG² group of formulae B and F is a suitable leaving group, as defined and described herein. In certain embodiments, the LG² group of formulae B and F is halogen, —OMs, —OTs, or —OTf. Examples of the LG² group of formulae B and F include —Br, —I, —OMs, —OTs, and —OTf.

The LG³ group of formula F is a suitable leaving group, as defined and described herein. In certain embodiments, the LG³ group of formula F is halogen, —OR,

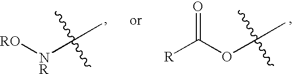

wherein each R is independently hydrogen or an optionally substituted group selected from C₁₋₆ aliphatic, 6-10 membered aryl, or 5-10 membered heteroaryl having 1-4 heteroatoms independently selected from nitrogen, oxygen or sulfur. Examples of LG³ groups of formula F include —OH, —OMe, —OEt, —Cl, —Br,

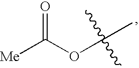

and the like. In some embodiments, LG³ is —Cl.

In certain embodiments, LG³ is —OH, and the reaction between a compound of formula F and a compound of formula B is carried out using suitable peptide coupling conditions. Suitable peptide coupling conditions are well known in the art and include those described in detail in Han et al., Tetrahedron, 60, 2447-67 (2004), the entirety of which is hereby incorporated by reference. In certain embodiments, the peptide coupling conditions include the addition of HOBt, DMAP, BOP, HBTU, HATU, BOMI, DCC, EDC, IBCF, or a combination thereof.

In certain embodiments, the compound of formula F is selected from 5-bromovaleryl chloride or 5-iodovaleryl chloride. In some embodiments, the compound of formula F is 5-bromovaleryl chloride.

One of ordinary skill in the art will appreciate that a variety of suitable leaving groups LG³ can be used to facilitate the reaction described in step S-5, and all such suitable leaving groups are contemplated by the present invention.

Suitable solvents for step S-5 include aprotic solvents, aliphatic halides, substituted and unsubstituted aromatic hydrocarbons, aliphatic ethers or combinations thereof. In certain embodiments, the solvent is selected from diethyl ether, t-butyl methyl ether, THF, ethyl acetate, DMSO, DMF, NMP, acetonitrile, dichloromethane, benzene, toluene, or combination thereof. In some embodiments, the solvent is a mixture of acetonitrile and DMF. In certain embodiments, the solvent is a 9:1 mixture of acetonitrile:DMF.

Suitable bases for step S-5 include tertiary amines such as pyridine, N-methylmorpholine, 1,4-diazabicyclo[2.2.2]octane, triethylamine (TEA), 1,8-diazabicyclo[5.4.0]undec-7-ene, diisopropylethylamine, and tetramethylethylenediamine; potassium carbonate, sodium bicarbonate, sodium carbonate, potassium hydroxide, sodium hydroxide, tetrabutylammonium hydroxide, benzyltrimethylammonium hydroxide, triethylbenzylammonium hydroxide, 1,1,3,3-tetramethylguanidine, and combinations thereof. In certain embodiments, the base is diisopropylethylamine.

Suitable temperatures at which the reaction described in step S-5 may occur include about −20° C. to about 60° C. In certain embodiments, the temperature is about −10° C. to about 25° C. In some embodiments, the temperature is about −10° C.

While not wishing to be bound by any particular theory, it is believed that the order of reagent addition may be useful in reducing the formation of byproducts in step S-5. In certain embodiments a compound of formula F is added slowly to a compound of formula C.

It will be appreciated that certain reaction conditions may result in the formation of a regioisomer in step S-5 (i.e., reaction of endocyclic pyrazole nitrogen with a compound of formula F). In certain embodiments, the product of step S-5 is treated to remove the undesired regioisomer. In some embodiments, the product of step S-5 is re-slurried with a particular solvent or solvents to remove the regioisomer. In some embodiments, the product of step S-5 is re-slurried with t-butyl methyl ether to remove the regioisomer.

At step S-6, a compound of formula B is reacted in a suitable solvent with N-acetylhomopiperazine, optionally in the presence of a suitable base and/or catalyst, to produce compound A, 5-(4-acetyl-1,4-diazepan-1-yl)-N-(5-(4-methoxyphenyl)-1H-pyrazol-3-yl)pentanamide. One of ordinary skill in the art will recognize that when a compound of formula B is 5-bromo-N-(5-(4-methoxyphenyl)-1H-pyrazol-3-yl)pentanamide, 5-iodo-N-(5-(4-methoxyphenyl)-1H-pyrazol-3-yl)pentanamide may be generated in situ by the addition of an iodide source, affording exchange of an iodine atom with the bromine atom. Examples of such iodide sources include, but are not limited to, sodium iodide, potassium iodide, hydrogen iodide, tetralkylammonium iodides, or mixtures thereof. While not wishing to be bound by any particular theory, it is believed that 5-iodo-N-(5-(4-methoxyphenyl)-1H-pyrazol-3-yl)pentanamide may be a more reactive species in step S-6 than 5-bromo-N-(5-(4-methoxyphenyl)-1H-pyrazol-3-yl)pentanamide, due to the greater leaving group character of iodide over bromide.

Suitable solvents for step S-6 include aprotic solvents, aliphatic halides, aliphatic ethers or combinations thereof. In certain embodiments, the solvent is selected from dichloromethane, diethyl ether, acetonitrile, THF, NMP, N-methyl morpholine, dimethylacetaminde, acetone, or combination thereof. In some embodiments, the solvent is acetone.

Suitable bases for step S-6 include tertiary amines such as pyridine, N-methylmorpholine, 1,4-diazabicyclo[2.2.2]octane, triethylamine (TEA), 1,8-diazabicyclo[5.4.0]undec-7-ene, diisopropylethylamine, and tetramethylethylenediamine; potassium carbonate, sodium bicarbonate, sodium carbonate, potassium hydroxide, sodium hydroxide, tetrabutylammonium hydroxide, benzyltrimethylammonium hydroxide, triethylbenzylammonium hydroxide, 1,1,3,3-tetramethylguanidine, and combinations thereof. In certain embodiments, the base is potassium carbonate. In certain embodiments, the potassium carbonate is milled. In certain embodiments, the base is diisopropylethylamine.

In certain embodiments, the iodide source used in step S-6 is sodium iodide. In certain embodiments, the iodide source is potassium iodide. In some embodiments, the potassium iodide is milled. In some embodiments, the iodide source is used in catalytic amounts ranging from about 0.1 to about 0.5 equivalents relative to a compound of formula B. In other embodiments, the iodide source is used stoichiometrically relative to a compound of formula B.

Suitable temperatures at which the reaction described in step S-6 may occur include about −20° C. to about 60° C. In certain embodiments, the temperature is about −10° C. to about 25° C. In some embodiments, the temperature is about 25° C.

In certain embodiments, as described in Example 5, an extractive workup step may be performed following step S-6. In certain embodiments, the organic phase comprises one or more organic solvents. In some embodiments, the organic solvents are ethanol, methyl tetrahydrofuran, dichloromethane, or a combination thereof. In certain embodiments, the solvents are ethanol and methyl tetrahydrofuran. In certain embodiments, the solvents are ethanol and dichloromethane. In some embodiments, the organic solvents are 5% ethanol in methyl tetrahydrofuran. In some embodiments, the organic solvents are 5% ethanol in dichloromethane.

In certain embodiments, dimer compounds may be formed as side products at step S-6. In such cases, it has been found that performing additional washes following HCl salt formation may be useful for reducing the presence of such dimer products.

At step S-7, compound A is reacted with hydrogen chloride, or an equivalent thereof, to form compound 1,5-(4-acetyl-1,4-diazepan-1-yl)-N-(5-(4-methoxyphenyl)-1H-pyrazol-3-yl)pentanamide hydrogen chloride.

Suitable solvents for step S-7 include polar solvents such as C₁ to C₄ alcohols (e.g. ethanol, methanol, 2-propanol), water, acetone or combinations thereof. In certain embodiments, the solvent is selected from ethanol, water, acetone, or combination thereof. In some embodiments, the solvent a mixture of acetone, water, and ethanol.

It will be appreciated that various types of absolute or denatured ethanol may be used in accordance with the present invention. In some embodiments, the ethanol is ethanol 1 L (denatured with 9% acetone). In some embodiments, the ethanol is ethanol 2B (denatured with 0.5% toluene).

One of ordinary skill in the art will appreciate that a number of suitable forms of hydrogen chloride can be used in step S-7 to produce the desired hydrochloride salt. Examples of such suitable forms include hydrogen chloride gas, aqueous solutions of hydrogen chloride, and solutions of hydrogen chloride in aliphatic ethers. In certain embodiments, the HCl is provided as an aqueous solution in acetone.

In certain embodiments, the number of equivalents of HCl used relative to compound of formula A is about 0.5-1.1 equivalents. In certain embodiments, the number of equivalents of HCl used relative to compound of formula A is about 0.8-1.0 equivalents. In some embodiments, the number of equivalents of HCl used relative to compound of formula A is about 0.93 equivalents.

It will be appreciated that the addition of excess HCl in step S-7 may result in the formation of a di-HCl salt. In certain embodiments, formation of such di-HCl salts is minimized or avoided by the addition of HCl in equivalents as described herein. In certain embodiments, formation of such di-HCl salts is minimized or avoided by careful, controlled addition of HCl.

Suitable temperatures at which the reaction described in step S-7 may occur include about −20° C. to about 60° C. In certain embodiments, the temperature is about −10° C. to about 35° C. In some embodiments, the temperature is about 25° C. to about 30° C.

As exemplified in Examples 3, 5 and 8, and without wishing to be bound by any particular theory, it is believed that the ternary solvent system of acetone, water, and ethanol, along with the number of equivalents of HCl used relative to compound of formula A, may be useful for obtaining the desired polymorph form I of compound I.

In certain embodiments, each of the aforementioned synthetic steps may be performed sequentially with isolation of each intermediate D, C, B, and A performed after each step. Alternatively, each of steps S-3, S-4, S-5, S-6 and S-7, as depicted in Scheme 2 above, may be performed in a manner whereby no isolation of one or more intermediates D, C, B, and A is performed. In certain embodiments, steps S-5, S-6, and S-7 are performed in sequence without any isolation of intermediates. In certain embodiments, steps S-6 and S-7 are performed in sequence without any isolation of intermediates. While not wishing to be bound by any particular theory, it is believed that such techniques may be useful in obtaining compounds of formulae B, A, and I in greater yield and purity.

In certain embodiments, compound A can be prepared according to Scheme 3 set forth below:

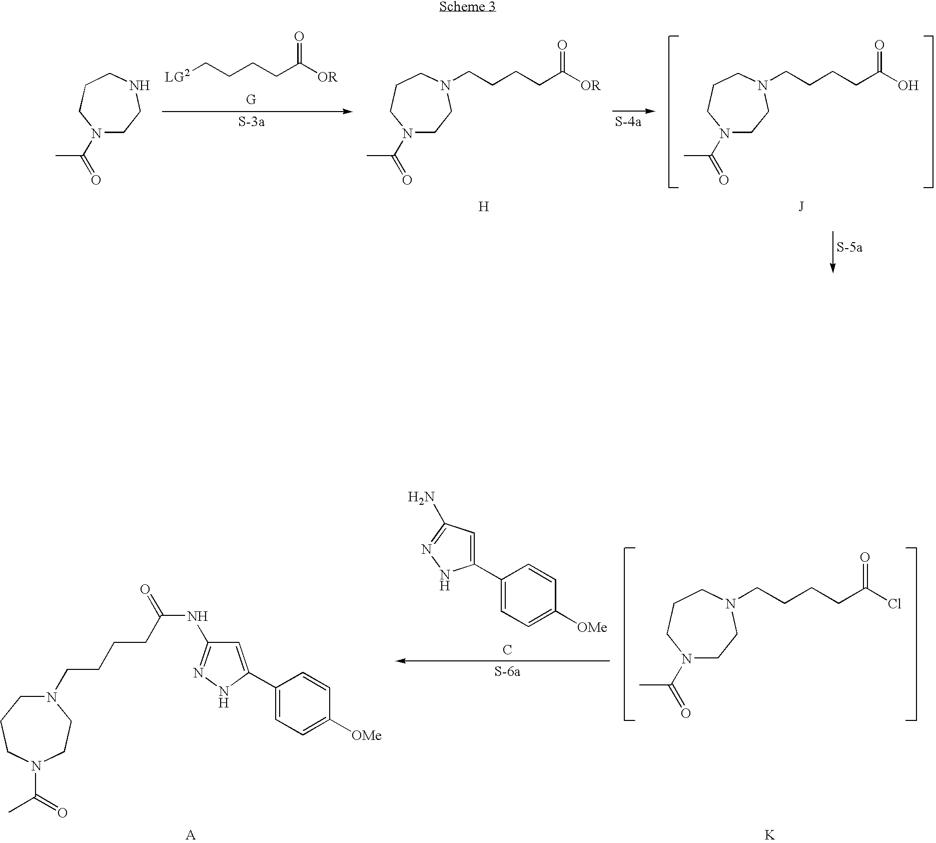

wherein K is as defined above.

At step S-3a, an ester of formula G is coupled with N-acetylhomopiperazine to form an ester of formula H. In certain embodiments, R is optionally substituted group selected from C₁₋₆ aliphatic. In some embodiments, R is C₁₋₃ aliphatic. In some embodiments, R is methyl.

A suitable base may be used to facilitate the reaction between a compound of formula G and N-acetylhomopiperazine. Examples of such bases include pyridine, diisopropylethylamine, triethylamine, sodium bicarbonate, sodium carbonate, potassium carbonate, and combinations thereof. In certain embodiments, the base is potassium carbonate.

One of ordinary skill in the art will recognize that when the LG² group of a compound of formula G is bromine, the iodo analog may be generated in situ by the addition of an iodide source, affording exchange of an iodine atom with the bromine atom. Examples of such iodide sources include, but are not limited to, sodium iodide, potassium iodide, hydrogen iodide, tetralkylammonium iodides, or mixtures thereof.

In certain embodiments, the iodide source is sodium iodide. In certain embodiments, the iodide source is potassium iodide. In some embodiments, the iodide source is used in catalytic amounts ranging from about 0.1 to about 0.5 equivalents relative to a compound of formula G. In other embodiments, the iodide source is used stoichiometrically relative to a compound of formula G.

In certain embodiments, step S-3a is carried out in the presence of a suitable solvent. In certain embodiments, the solvent is selected from a polar aprotic solvent. Exemplary solvents include dichloromethane, diethyl ether, acetonitrile, THF, NMP, N-methyl morpholine, dimethylacetamide, acetone, or combination thereof. In some embodiments, the solvent is acetone.

At step S-4a, an ester of formula H is saponified with a suitable acid or base to provide carboxylic acid J. One of ordinary skill in the art will be aware of appropriate acids and bases that may be used. Such suitable bases include strong inorganic bases i.e., those that completely dissociate in water under formation of hydroxide anion. Examples of such bases include alkaline metals, alkaline earth metal hydroxides, and combinations thereof. In some embodiments, a suitable acid is a Lewis acid.

At step S-5a, carboxylic acid J is chlorinated to form acyl chloride K. In certain embodiments, the chlorination is facilitated by POCl₃. In certain embodiments, step S-5a is carried out in the presence of a suitable solvent. In certain embodiments, the solvent is selected from a polar aprotic solvent. Exemplary solvents include dichloromethane, diethyl ether, acetonitrile, THF, NMP, N-methyl morpholine, dimethylacetamide, acetone, or combination thereof. In some embodiments, the solvent is dimethylacetamide.

At step S-6a, acyl chloride K is reacted with aryl aminopyrazole C to provide compound A. Compound A may then be used as described above and herein.

In certain embodiments, the present invention provides a method for preparing compound I-1′:

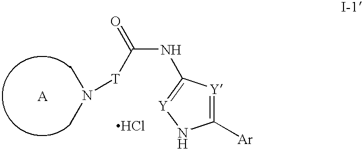

wherein each of Ar, Y, Y′, T, and Ring A is as defined above and herein;
comprising the steps of:
(a) providing compound I-1:

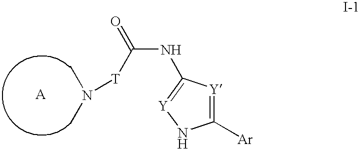

and
(b) treating said compound I-1 with hydrochloric acid to form compound I-1′.

One of ordinary skill in the art will appreciate that a number of suitable forms of hydrogen chloride can be used to produce the desired hydrochloride salt. Examples of such suitable forms include hydrogen chloride gas, aqueous solutions of hydrogen chloride, and solutions of hydrogen chloride in aliphatic ethers. In certain embodiments, the HCl is provided as an aqueous solution in acetone. In certain embodiments, compound A is treated with hydrochloric acid in a solvent mixture of acetone, water, and ethanol.

In certain embodiments, the number of equivalents of HCl used relative to compound of formula I-1 is about 0.5-1.1 equivalents. In certain embodiments, the number of equivalents of HCl used relative to compound of formula I-1 is about 0.8-1.0 equivalents. In some embodiments, the number of equivalents of HCl used relative to compound of formula I-1 is about 0.93 equivalents.

In certain embodiments, the present invention provides a method for preparing compound I-1:

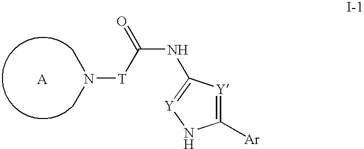

wherein each of Ar, Y, Y′, T, and Ring A is as defined above and herein,
comprising the steps of:
(a) providing compound B′:

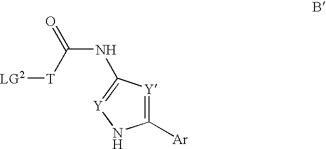

wherein, LG² is a suitable leaving group, and
(b) treating said compound of formula B′ with a compound of formula G′:

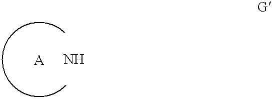

optionally in the presence of a suitable base and/or additive, to form compound I-1.

The LG² group of formula B′ is a suitable leaving group, as defined and described herein. Suitable leaving groups are well known in the art and include, but are not limited to, halogen, —OMs, —OTs, and —OTf. Examples of the LG² group of formula B′ include Br, I, —OMs, —OTs, and —OTf.

One of ordinary skill in the art will recognize that when a compound of formula B′ is 5-bromo-N-(5-(4-methoxyphenyl)-1H-pyrazol-3-yl)pentanamide, 5-iodo-N-(5-(4-methoxyphenyl)-1H-pyrazol-3-yl)pentanamide may be generated in situ by the addition of an iodide source, affording exchange of an iodine atom with the bromine atom. Examples of such iodide sources include, but are not limited to, sodium iodide, potassium iodide, hydrogen iodide, tetralkylammonium iodides, or mixtures thereof.

In certain embodiments, the iodide source is sodium iodide. In certain embodiments, the iodide source is potassium iodide. In some embodiments, the iodide source is used in catalytic amounts ranging from about 0.1 to about 0.5 equivalents relative to a compound of formula B′. In other embodiments, the iodide source is used stoichiometrically relative to a compound of formula B′.

A suitable base may be used to facilitate the reaction between a compound of formula B′ and a compound of formula G′. Examples of such bases include pyridine, diisopropylethylamine, triethylamine, sodium bicarbonate, sodium carbonate, potassium carbonate, and combinations thereof. In certain embodiments, the base is diisopropylethylamine. In certain embodiments, the base is potassium carbonate.

In certain embodiments, the transformation of a compound of formula B′ to compound I-1 is performed in the presence of a suitable solvent. In certain embodiments, the solvent is selected from a polar aprotic solvent. Exemplary solvents include dichloromethane, diethyl ether, acetonitrile, THF, NMP, N-methyl morpholine, dimethylacetaminde, acetone, or combination thereof. In some embodiments, the solvent is acetone.

According to another embodiment, the present invention provides a method for preparing a compound of formula B′:

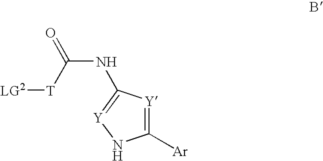

wherein each of Ar, Y, Y′, T, LG² and Ring A is as defined above and herein comprising the steps of:
(a) providing compound C:

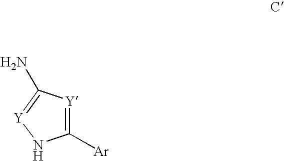

and
(b) treating said compound C′ in the presence of a suitable base with a compound of formula F′:

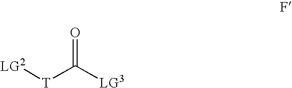

wherein LG³ is a suitable leaving group, to form a compound of formula B′.

The LG² group of formulae B′ and F′ is a suitable leaving group, as defined and described herein. Suitable leaving groups are well known in the art and include, but are not limited to, halogen, —OMs, —OTs, and —OTf. In certain embodiments, the LG² group of formulae B′ and F′ include Br, I, —OMs, —OTs, and —OTf.

The LG³ group of formula F′ is a suitable leaving group, and defined and described herein. Suitable leaving groups are well known in the art and include, but are not limited to, halogen, —OR,

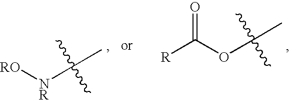

wherein each R is independently hydrogen or an optionally substituted group selected from C₁₋₆ aliphatic, 6-10 membered aryl, or 5-10 membered heteroaryl having 1-4 heteroatoms independently selected from nitrogen, oxygen or sulfur. Examples of LG³ groups of formula F′ include —OH, —OMe, —OEt, —Cl, —Br,

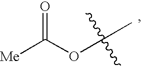

and the like. In some embodiments, LG³ is —Cl.

In certain embodiments, the compound of formula F′ is selected from 5-bromovaleryl chloride or 5-iodovaleryl chloride. In some embodiments, the compound of formula F′ is 5-bromovaleryl chloride.

Suitable bases to facilitate the transformation of compound C′ to a compound of formula B′ include pyridine, diisopropylethylamine, triethylamine, sodium bicarbonate, sodium carbonate, and combinations thereof. In certain embodiments, the base is diisopropylethylamine.

In certain embodiments, the transformation of compound C′ to a compound of formula B′ is carried out in the presence of a suitable solvent. In certain embodiments, the solvent is selected from diethyl ether, t-butyl methyl ether, THF, ethyl acetate, DMSO, DMF, NMP, acetonitrile, dichloromethane, benzene, toluene, or combination thereof. In some embodiments, the solvent is a mixture of acetonitrile and DMF. In certain embodiments, the solvent is a 9:1 mixture of acetonitrile:DMF.

In certain embodiments, the present invention provides a method for preparing compound I:

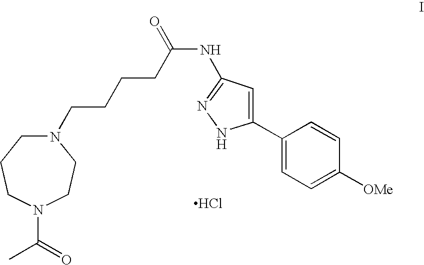

comprising the steps of:
(a) providing compound A:

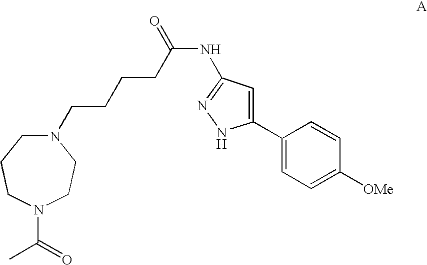

and
(b) treating said compound A with hydrochloric acid to form compound I.

One of ordinary skill in the art will appreciate that a number of suitable forms of hydrogen chloride can be used to produce the desired hydrochloride salt. Examples of such suitable forms include hydrogen chloride gas, aqueous solutions of hydrogen chloride, and solutions of hydrogen chloride in aliphatic ethers. In certain embodiments, the HCl is provided as an aqueous solution in acetone. In certain embodiments, compound A is treated with hydrochloric acid in a solvent mixture of acetone, water, and ethanol.

In certain embodiments, the number of equivalents of HCl used relative to compound of formula A is about 0.5-1.1 equivalents. In certain embodiments, the number of equivalents of HCl used relative to compound of formula A is about 0.8-1.0 equivalents. In some embodiments, the number of equivalents of HCl used relative to compound of formula A is about 0.93 equivalents.

In certain embodiments, the present invention provides a method for preparing compound A:

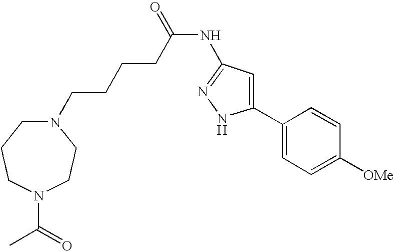

comprising the steps of:
(a) providing compound B:

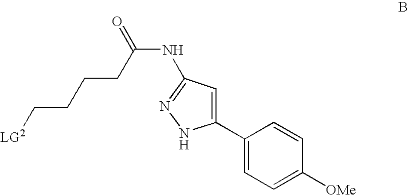

wherein, LG² is a suitable leaving group, and
(b) treating said compound of formula B with N-acetylhompiperazine, optionally in the presence of a suitable base and/or additive, to form compound A.

The LG² group of formula B is a suitable leaving group. Suitable leaving groups are well known in the art and include, but are not limited to, halogen, —OMs, —OTs, and —OTf. Examples of the LG² group of formula B include —Br, —I, —OMs, —OTs, and —OTf.

One of ordinary skill in the art will recognize that when a compound of formula B is 5-bromo-N-(5-(4-methoxyphenyl)-1H-pyrazol-3-yl)pentanamide, 5-iodo-N-(5-(4-methoxyphenyl)-1H-pyrazol-3-yl)pentanamide may be generated in situ by the addition of an iodide source, affording exchange of an iodine atom with the bromine atom. Examples of such iodide sources include, but are not limited to, sodium iodide, potassium iodide, hydrogen iodide, tetralkylammonium iodides, or mixtures thereof.

In certain embodiments, the iodide source is sodium iodide. In certain embodiments, the iodide source is potassium iodide. In some embodiments, the iodide source is used in catalytic amounts ranging from about 0.1 to about 0.5 equivalents relative to a compound of formula B. In other embodiments, the iodide source is used stoichiometrically relative to a compound of formula B.

A suitable base may be used to facilitate the reaction between a compound of formula B and N-acetylhompiperazine. Examples of such bases include pyridine, diisopropylethylamine, triethylamine, sodium bicarbonate, sodium carbonate, potassium carbonate, and combinations thereof. In certain embodiments, the base is diisopropylethylamine. In certain embodiments, the base is potassium carbonate.

In certain embodiments, the transformation of a compound of formula B to compound A is performed in the presence of a suitable solvent. In certain embodiments, the solvent is selected from a polar aprotic solvent. Exemplary solvents include dichloromethane, diethyl ether, acetonitrile, THF, NMP, N-methyl morpholine, dimethylacetaminde, acetone, or combination thereof. In some embodiments, the solvent is acetone.

According to another embodiment, the present invention provides a method for preparing a compound of formula B:

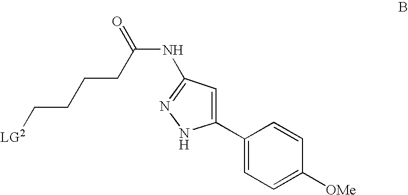

comprising the steps of:
(a) providing compound C:

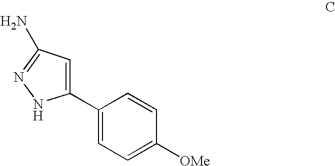

and
(b) treating said compound C in the presence of a suitable base with a compound of formula F:

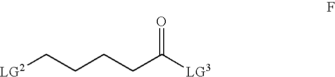

to form a compound of formula B.

The LG² group of formulae B and F is a suitable leaving group. Suitable leaving groups are well known in the art and include, but are not limited to, halogen, —OMs, —OTs, and —OTf. Examples of the LG² group of formulae B and F include —Br, —I, —OMs, —OTs, and —OTf.

The LG³ group of formula F is a suitable leaving group. Suitable leaving groups are well known in the art and include, but are not limited to, halogen, —OR,

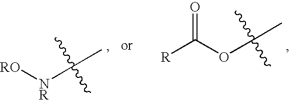

wherein each R is independently hydrogen or an optionally substituted group selected from C₁₋₆ aliphatic, 6-10 membered aryl, or 5-10 membered heteroaryl having 1-4 heteroatoms independently selected from nitrogen, oxygen or sulfur. Examples of LG³ groups of formula F include —OH, —OMe, —OEt, —Cl, —Br,

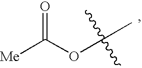

and the like. In some embodiments, LG³ is —Cl.

In certain embodiments, the compound of formula F is selected from 5-bromovaleryl chloride or 5-iodovaleryl chloride. In some embodiments, the compound of formula F is 5-bromovaleryl chloride.

Suitable bases to facilitate the transformation of compound C to a compound of formula B include pyridine, diisopropylethylamine, triethylamine, sodium bicarbonate, sodium carbonate, and combinations thereof. In certain embodiments, the base is diisopropylethylamine.

In certain embodiments, the transformation of compound C to a compound of formula B is carried out in the presence of a suitable solvent. In certain embodiments, the solvent is selected from diethyl ether, t-butyl methyl ether, THF, ethyl acetate, DMSO, DMF, NMP, acetonitrile, dichloromethane, benzene, toluene, or combination thereof. In some embodiments, the solvent is a mixture of acetonitrile and DMF. In certain embodiments, the solvent is a 9:1 mixture of acetonitrile:DMF.

In certain embodiments, the present invention provides a method for preparing compound C:

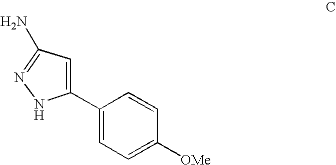

comprising the steps of:
(a) providing compound D:

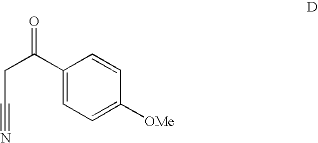

and
(b) treating said compound of formula D with hydrazine, or an equivalent thereof, to form compound C.

In certain embodiments, the hydrazine equivalent used is a hydrate. In some embodiments, the hydrazine equivalent is a monohydrate.

In certain embodiments, the transformation of compound D to compound C is carried out in the presence of a suitable solvent. In certain embodiments, the solvent is selected from ethanol, toluene, dichloromethane, diethyl ether, THF, benzene or combination thereof. In some embodiments, the solvent is ethanol.

In another embodiment, the present invention provides a method for preparing compound D:

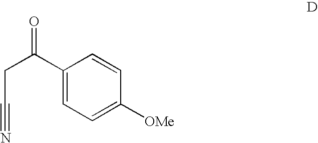

comprising the steps of:
(a) combining a compound of formula E:

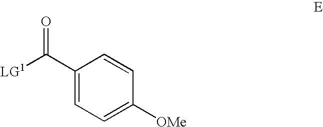

wherein, LG¹ is a leaving group, with acetonitrile to form a mixture thereof, and;
(b) treating said mixture with a suitable base to give compound D.

In certain embodiments, the base is selected the group consisting of NaH, LDA, NaHMDS, LHMDS, KHMDS, potassium t-amylate, BuLi, and NaOtBu. In some embodiments, the base is LHMDS.

The acetonitrile used in step (a) may be used in a range of equivalents relative to aromatic ester E. In certain embodiments, the equivalents of acetonitrile range from 0.5 to 50 equivalents. In certain embodiments, the equivalents of acetonitrile range from 2 to 10 equivalents. In some embodiments, the equivalents of acetonitrile range from 4 to 6 equivalents.

In certain embodiments, the transformation of a compound of formula E to compound D may be carried out in the presence of a suitable solvent. In some embodiments, the solvent is selected from toluene, acetonitrile, diethyl ether, t-butyl methyl ether, THF, benzene, dichloromethane or combinations thereof. In certain embodiments, no solvent is used.

According to another embodiment, the present invention provides compound I substantially free of N-acetylhompiperazine, 5-iodovaleryl chloride, 5-bromovaleryl chloride, 5-chlorovaleryl chloride, and compounds F-1, F-2, and F-3:

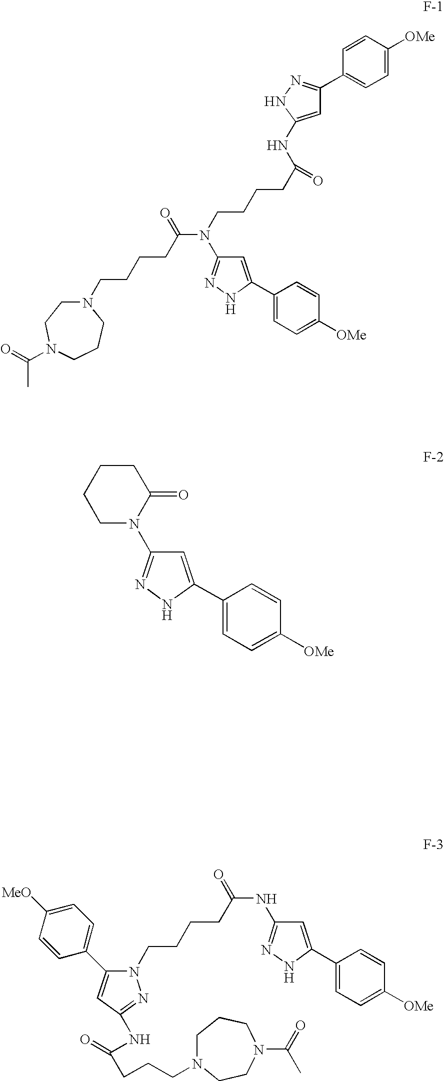

Compounds F-1, F-2, and F-3 are impurities that may arise from steps S-1, S-2, S-5, or S-6 as described above and herein. “Substantially free,” as used herein, means that at least about 80% by weight of the desired compound is present. In other embodiments, at least about 92% by weight of a desired compound is present. In still other embodiments of the invention, at least about 99% by weight of a desired compound is present. Such impurities may be isolated from product mixtures by any method known to those skilled in the art, including liquid chromatography (LC).

In some embodiments, the present invention provides compound I having total impurities of less than 0.5%, less than 0.4%, or less than 0.3% by weight.

The present invention provides methods that provide compound I in substantially higher yields than described previously (U.S. Patent Application Ser. No. 60/880,629, filed Jan. 16, 2007).

As will be readily apparent to one skilled in the art, the unsubstituted ring nitrogen pyrazoles, as in the compounds of the present invention, are known to rapidly equilibrate in solution, as mixtures of both tautomers:

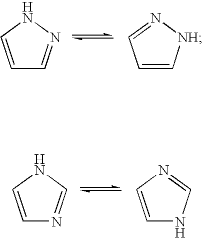

for the compounds described above and herein, where only one tautomer is indicated, the other tautomer is also intended as within the scope of the present invention. Certain compound names may indicate one tautomer. For the compounds named above and herein, where only one tautomer is indicated by the compound name, the other tautomer is also intended as within the scope of the present invention.

Compounds of the invention can be in the form of free bases or acid addition salts, preferably salts with pharmaceutically acceptable acids.

Pharmacological activity of a representative group of compounds of formula I-1 was demonstrated in an in vitro assay utilizing cells stably transfected with the alpha 7 nicotinic acetylcholine receptor and cells expressing the alpha 1 and alpha 3 nicotinic acetylcholine receptors and 5HT₃ receptor as controls for selectivity.

Compounds of formula I-1 may be provided according to the present invention in any of a variety of useful forms, for example as pharmaceutically acceptable salts, as particular crystal forms, etc. In some embodiments, prodrugs of compounds of formula I-1 are provided. Various forms of prodrugs are known in the art, for example as discussed in Bundgaard (ed.), Design of Prodrugs, Elsevier (1985); Widder et al. (ed.), Methods in Enzymology, vol. 4, Academic Press (1985); Kgrogsgaard-Larsen et al. (ed.); “Design and Application of Prodrugs”, Textbook of Drug Design and Development, Chapter 5, 113-191 (1991); Bundgaard et al., Journal of Drug Delivery Reviews, 8:1-38 (1992); Bundgaard et al., J. Pharmaceutical Sciences, 77:285 et seq. (1988); and Higuchi and Stella (eds.), Prodrugs as Novel Drug Delivery Systems, American Chemical Society (1975).

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

### EXEMPLIFICATION

### Experimental Procedures

### Synthesis of Compounds

### General

Unless otherwise specified all nuclear magnetic resonance spectra were recorded using a Varian Mercury Plus 400 MHz spectrometer equipped with a PFG ATB Broadband probe. HPLC-MS analyses were performed with a Waters 2795 separation module equipped with a Waters Micromass ZQ (ES ionisation) and Waters PDA 2996, using a Waters XTerra MS C18 3.5 μm 2.1×50 mm column.

Preparative HLPC was run using a Waters 2767 system with a binary Gradient Module Waters 2525 pump and coupled to a Waters Micromass ZQ (ES) or Waters 2487 DAD, using a Supelco Discovery HS C18 5.0 μm 10×21.2 mm column Gradients were run using 0.1% formic acid/water and 0.1% formic acid/acetonitrile with gradient 5/95 to 95/5 in the run time indicated in the Examples.

All column chromatography was performed following the method of Still, C.; J. Org Chem 43, 2923 (1978). All TLC analyses were performed on silica gel (Merck 60 F254) and spots revealed by UV visualisation at 254 nm and KMnO4 or ninhydrin stain.

When specified for array synthesis, heating was performed on a Buchi Syncore® system. All microwave reactions were performed in a CEM Discover oven.

### Abbreviations Used Throughout the Experimental Procedures

AcOEt ethyl acetate
DCM dichloromethane
DCE 1,2-dichloroethane
DMEA N,N-dimethylethylamine
DMF N,N-dimethylformamide
DMSO, dmso dimethylsulphoxide
DMA N,N-dimethylacetamide
scx strong cation exchanger
TEA triethylamine
TFA trifluoroacetic acid
THF tetrahydrofuran
TLC thin layer chromatography
LC-MS liquid chromatography-mass spectrometry
HPLC high performance liquid chromatography

### General 3-amino-5-aryl/heteroaryl pyrazole synthesis

The 3-amino-5-aryl/heteroaryl pyrazoles used in the Examples were either commercially available or synthesised using the routes shown in the scheme below:

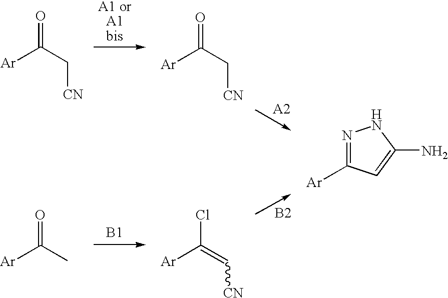

General procedure for aryl/heteroaryl β-ketonitrile synthesis (A1):

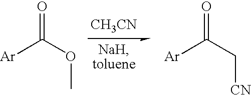

Aryl or heteroaryl methyl carboxylate were commercially available or were synthesized according to the following standard procedure: the aryl or heteroaryl carboxylic acid (32 mmol) was dissolved in MeOH (40 mL) and sulfuric acid (1 mL) was added. The mixture was refluxed overnight, after which the solvent was evaporated under reduced pressure; the crude was dissolved in DCM and washed with saturated aqueous NaHCO3 solution. The organic phase was dried and evaporated under reduced pressure, and the crude was used without further purification.

To a solution of an aryl or heteroaryl methyl carboxylate (6.5 mmol) in dry toluene (6 mL) under N₂, NaH (50-60% dispersion in mineral oil, 624 mg, 13 mmol) was carefully added. The mixture was heated at 80° C. and then dry CH₃CN was added dropwise (1.6 mL, 30.8 mmol). The reaction was heated for 18 hours and generally the product precipitated from the reaction mixture as Na salt.

The reaction was then allowed to cool down to room temperature and the solid formed was filtered and then dissolved in water. The solution was then acidified with 2N HCl solution and at pH between 2-6 (depending on the ring substitution on the aryl/heteroaryl system) the product precipitated and was filtered off. If no precipitation occurred, the product was extracted with DCM.

After work-up, the products were generally used in the following step without further purification. The general yield was between 40 and 80%.

General Procedure for Aryl/Heteroaryl β-Ketonitrile Synthesis (Route A1bis):

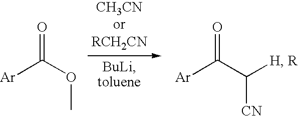

Aryl- or heteroaryl-carboxylic acid methyl esters are commercially available or were synthesized under the standard procedure, as described in general procedure A1.

To a solution of dry alkanenitrile in toluene (1 mmol/mL, 5 eq.) cooled down to −78° C. under nitrogen, a solution of n-butyllithium in n-hexane (1.6 N, 3.5 eq) was added dropwise. The mixture was left stirring at −78° C. for 20 minutes and then a solution of the aryl or heteroaryl methyl carboxylate in toluene (0.75 mmol/mL, 1 eq.) was added and the reaction allowed to reach room temperature. Upon reaction completion, after about 20 minutes, the mixture was cooled down to 0° C. and HCl 2 N was added to pH 2. The organic phase was recovered, dried over Na₂SO₄ and concentrated under reduced pressure, affording the title product which was generally used without further purification.

### General Procedure for Aryl Aminopyrazole Synthesis (Route A2):

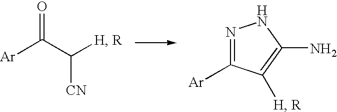

To a solution of the β-ketonitrile (7.5 mmoL), in absolute EtOH (15 mL) hydrazine monohydrate (0.44 mL, 9.0 mmol) was added and the reaction was heated at reflux for 18 hrs. The reaction mixture was allowed to cool to room temperature and the solvent was evaporated under reduced pressure. The residue was dissolved in DCM and washed with water.

The organic phase was concentrated under reduced pressure to give a crude product that was purified by SiO₂ column or by precipitation from Et₂O.

Yields were generally between 65 and 90%.

### Hydroxy-Aryl- or Hydroxy-Heteroaryl-Carboxylic Acid to Methyl Ester—General Procedure

4-hydroxy-benzoic acid (usually 24.0 mmol) was dissolved in MeOH (50 mL) and sulfuric acid (1 mL/g substrate) was added. The mixture was refluxed overnight, after which the solvent was evaporated under reduced pressure; the crude was dissolved in DCM and washed with saturated NaHCO₃ to basic pH. The organic phase was dried and evaporated under reduced pressure, and the product was used without further purification. The yields were between 80 and 90%.

### Hydroxy-Aryl- or Hydroxy-Heteroaryl-Carboxylic Acid Methyl Ester to F₂Cho-Aryl- or Heteroarylcarboxylic Acid Methyl Ester-General Procedure

Under a N₂ atmosphere, 4-hydroxy-benzoic acid methyl or ethyl ester (1.0 eq) and sodium chlorodifluoroacetate (1.2 eq) were dissolved in DMF (20-25 mL) in a two neck round bottom flask; potassium carbonate (1.2 eq) was added and the mixture was heated at 125° C. until complete conversion of the starting material was observed by LC-MS. The mixture was then diluted with water and extracted with DCM; the organic phase was dried and removed under reduced pressure, and the crude was purified through Si column to obtain the product (Yields from 20 to 70%).

The following Table 1 reports yields and analytical data obtained in the preparation of a series of F₂CHO-aryl- or F₂CHO-heteroaryl-carboxylic acid methyl esters prepared according to the general procedures described above

TABLE 1

| Starting | | |
|---|---|---|
| material | Methyl ester —OH | Methyl ester —OCHF2 |
| 3-Fluoro-4- | C₈H₇FO₃ | C₉H₇F₃O₃ |
| hydroxy- | Yield = 85% | Yield = 66% |
| benzoic acid | 1H NMR (DMSO-d6) δ | 1H NMR (DMSO-d6) δ 3.78 (3H, |
| | 3.78 (3H, s), 7.00-7.05 (1H, m), | s), 6.24 (1H, m), 7.61 (1H, m), |
| | 7.60-7.65 (2H, m) | 7.64 (1H, m), 10.89 (1H, bs) |
| 2,6-Difluoro-4- | C₈H₆F₂O₃ | C₉H₆F₄O₃ |
| hydroxy- | Yield = 85% | Yield = 34% |
| benzoic acid | 1H NMR (DMSO-d6) δ | 1H NMR (DMSO-d6) δ 3.86 (3H, |
| | 3.79 (s, 3H, s), 6.53 (2H, d, J = 10.8 Hz), | s), 7.18-7.24 (2H, m), 7.42 (1H, t, |
| | 11.13 (1H, s) | J = 72.4 Hz). |
| 3,5-Dichloro-4- | Commercially available | C₉H₆Cl₂F₂O₃ |
| hydroxy- | | Yield = 74% |
| benzoic acid | | 1H NMR (DMSO-d6) δ 3.31 (3H, |
| | | s), 7.22 (1H, t, J = 71.6 Hz), |
| | | 8.05 (2H, s). |
| 3-Chloro-4- | Commercially available | C₉H₇ClF₂O₃ |
| hydroxy- | | Yield = 85% |
| benzoic acid | | 1H NMR (DMSO-d6) δ 3.85 (3H, |
| | | s), 7.39 (1H, t, J = 72.4 Hz), |
| | | 7.50 (1H, t, J = 8.4 Hz), 7.82-7.89 (2H, |
| | | m). |
| 4-Hydroxy-3- | Commercially available | C₁₀H₁₀F₂O₄ |
| methoxy- | | Yield = 85% |
| benzoic acid | | 1H NMR (DMSO-d6) 3.84 (3H, |
| | | s), 3.87 (3H, s); 7.22 (1H, t, J = 73.6 Hz), |
| | | 7.29 (1H, d, J = 8.4 Hz), |
| | | 7.57-7.60 (2H, m). |
| 4-Hydroxy-2- | C₉H₁₀O₃ | C₁₀H₁₀F₂O₃ |
| methyl-benzoic | Yield = 95% | Yield = 85% |
| acid | 1H NMR (DMSO-d6) | 1H NMR (DMSO-d6) 2.52 (3H, br |
| | 2.43 (3H, br s), 3.72 (3H, s); | s), 3.80 (3H, s); 7.07-7.13 (2H, m); |
| | 6.61-6.64 (2H, m); 7.71-7.73 (1H, | 7.34 (1H, t, J = 73.6 Hz), 7.89 (1H, |
| | m), 10.10 (1H, s). | d, J = 8.8 Hz). |

### 3-Imidazo[1,2-a]pyridin-6-yl-3-oxo-propionitrile

The product was obtained starting from imidazo[1,2-a]pyridine-6-carboxylic acid methyl ester according to general procedure A1

Yield 39%

C₁₀H₇N₃O Mass (calculated) [185]; (found) [M+H⁺]=186 [M−H]=184

LC Rt=0.23, 100% (3 min method)

¹H-NMR: (dmso-d6): 4.72 (2H, s), 7.61-7.65 (2H, m), 7.70 (1H, m), 8.07 (1H, s), 9.40 (s, 1H).

### 5-Imidazo[1,2-a]pyridin-6-yl-1H-pyrazol-3-ylamine

The title compound was synthesized according to general procedure A2 starting from 3-imidazo[1,2-a]pyridin-6-yl-3-oxo-propionitrile

Yield: 84%

C₁₀H₉N₅ Mass (calculated) [199]; (found) [M+1]=200

LCMS, (5 min method, RT=0.21 min,

NMR (1H, 400 MHz, MeOH-d₄) 3.34 (s, 2H), 5.90 (br s, 1H), 7.57 (s, 1H), 7.63 (br s, 1H), 7.86 (s, 1H), 8.73 (s, 1H)

### Chlorocynnamonitrile Synthesis (Route B1)

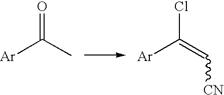

POC₃ (2 eq with respect to the aryl/heteroaryl acetophenone) were added dropwise to 4 molar equivalents of anhydrous DMF cooled down to 0° C., at such a rate that the temperature did not exceed 10° C. The acetophenone (1 eq) was then added dropwise and the reaction was allowed to reach room temperature.

The reaction was then stirred for further 30 min and then 0.4 mmol of hydroxylamine hydrochloride were added. The reaction was then heated up to 50° C., after which heating was removed and additional 4 eq. of hydroxylamine hydrochloride were added portionwise (at such a rate that the temperature never exceeded 120° C.). The reaction was then stirred until the temperature of the mixture spontaneously decreased to 25° C. Water (100 mL) were then added and the mixture was extracted with diethyl ether. The organic phase was dried over Na₂SO₄ and concentrated under reduced pressure. The crude product was used for the next step without further purification.

### Aryl Aminopyrazole Synthesis (Route B2)

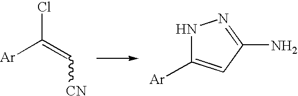

To a solution of the chlorocynnamonitrile (0.5 mmol/mL, 1 eq) in absolute EtOH 2 eq of hydrazine monohydrate were added and the reaction was heated at reflux for 4 hrs. The reaction mixture was allowed to cool to room temperature and the solvent was evaporated under reduced pressure. The residue was triturated with Et₂O, allowing to recover the title compound which was generally used without further purification.

### 5-(2-Trifluoromethyl-phenyl)-2H-pyrazol-3-ylamine

### a) 3-Oxo-3-(2-trifluoromethyl-phenyl)-propionitrile

The product was prepared according to the general procedure for aminopyrazole synthesis (route A1) from 2-trifluoromethyl-benzoic acid methyl ester (3.1 g, 14.0 mmol, 1.0 eq). The crude was precipitated from HCl to give the title product as a yellow solid (2.8 g, yield: 94%).

C₁₀H₆F₃NO

¹H-NMR (CD₃OD): 4.90 (2H, br s); 7.52-7.86 (4H, m).

### b) 5-(2-Trifluoromethyl-phenyl)-2H-pyrazol-3-ylamine

The product was prepared according to general procedure for aminopyrazole synthesis (route A2). The crude was purified through Si column (eluent: DCM) and dried to give the title product (0.6 g, 20% Yield).

C₁₀H₈F₃N₃

### 5-(2,6-Dimethyl-phenyl)-2H-pyrazol-3-ylamine

### a) 3-(2,6-Dimethyl-phenyl)-3-oxo-propionitrile

The product was prepared according to the general procedure for aminopyrazole synthesis (route A1), refluxing the mixture overnight and then for 2 h at 110° C. The crude product was extracted with DCM and used in the following step without further purification (2.2 g, yield: 76%).

C₁₁H₁₁NO

### b) 5-(2,6-Dimethyl-phenyl)-2H-pyrazol-3-ylamine

The product was prepared according to general procedure for aminopyrazole synthesis (route A2). The crude was purified through Si column (eluent: DCM) and washed with water, extracted and dried to give the title product (0.25 g, yield 10%).

### C₁₁H₁₃N₃

¹H-NMR (CD₃OD): 2.09-2.23 (6H, m); 7.04-7.12 (2H, m); 7.18-7.26 (2H, m).

### 5-(2-Chloro-4-fluoro-phenyl)-2H-pyrazol-3-ylamine

### a) 3-(2-Chloro-4-fluoro-phenyl)-3-oxo-propionitrile

The product was prepared according to the general procedure for aminopyrazole synthesis (route A1) from 2-chloro-4-fluoro-benzoic acid methyl ester (0.7 g, 3.7 mmol, 1.0 eq). The crude product was extracted with DCM and used in the following step without further purification (0.4 g, yield: 60%).

C₉H₅ClFNO

### b) 5-(2-Chloro-4-fluoro-phenyl)-2H-pyrazol-3-ylamine

The product was prepared according to general procedure for aminopyrazole synthesis (route A2). The crude was dissolved in DCM, washed with sat NaHCO₃, extracted and dried to give the title product (0.12 g, yield 26%).

C₉H₇ClFN₃

¹H-NMR (dmso-d6): 7.03-7.53 (4H, m).

### 5-(5-tert-Butyl-thiophen-2-yl)-2H-pyrazol-3-ylamine

### a) 3-(5-tert-Butyl-thiophen-2-yl)-3-oxo-propionitrile

The product was prepared according to the general procedure for aminopyrazole synthesis (route A1) from 5-tert-Butyl-thiophene-2-carboxylic acid methyl ester (3.0 g, 15.0 mmol, 1.0 eq). The crude product was extracted with DCM and used in the following step without further purification (2.7 g, yield: 86%).

C₁₁H₁₃NOS

### b) 5-(5-tert-Butyl-thiophen-2-yl)-2H-pyrazol-3-ylamine

The product was prepared according to general procedure for aminopyrazole synthesis (route A2). The crude was washed with water and precipitated to give the title product (2.7 g, yield 91%).

C₁₁H₁₅N₃S

Mass (calculated) [221]; (found) [M+H⁺]=222.

LC Rt=2.53 min, 94% (10 min method)

¹H-NMR (dmso-d6): 1.26-1.29 (9H, m); 4.87 (2H, br s); 5.47 (1H, br s); 6.66-6.79 (1H, m); 6.97-7.02 (1H, m)

### 5-(3-Chloro-2-methyl-phenyl)-2H-pyrazol-3-ylamine

### a) 2-Ethyl-benzoic acid methyl ester

2-Ethyl-benzoic acid (3.0 g, 17.6 mmol) was dissolved in MeOH (20 mL) and sulfuric acid (1 mL) was added. The mixture was refluxed overnight, after which the solvent was evaporated under reduced pressure; the crude was dissolved in DCM and washed with saturated Na₂CO₃ to basic pH. The organic phase was dried and evaporated under reduced pressure, and the product (3.1 g, yield 96%) was used without further purification

C₉H₉ClO₂

¹H-NMR (dmso-d6): 2.48 (3H, br s); 3.82 (3H, s); 7.31 (1H, t, J=7.6 Hz); 7.63-7.67 (2H, m).

### b) 3-(3-Chloro-2-methyl-phenyl)-3-oxo-propionitrile

The product was prepared according to the general procedure for aminopyrazole synthesis (route A1) from 3-Chloro-2-methyl-benzoic acid methyl ester (3.1 g, 16.8 mmol, 1.0 eq). The crude product was precipitated form water and used in the following step without further purification (2.4 g, yield: 74%).

C₁₀H₈ClNO

¹H-NMR (dmso-d6): 2.31 (3H, br s); 4.64 (2H, br s); 7.27-7.36 (2H, m); 7.54-7.77 (1H, m).

### c) 5-(3-Chloro-2-methyl-phenyl)-2H-pyrazol-3-ylamine

The product was prepared according to general procedure for aminopyrazole synthesis (route A2). The crude product was purified through SiO₂ column (20 g) with gradient elution from 100% EtOAc to EtOAc-MeOH 80:20. The title product (1.3 g, yield 50%) was obtained.

C₁₀H₁₀ClN₃

Mass (calculated) [207]; (found) [M+H⁺]=208.

LC Rt=1.96 min, 85% (10 min method)

¹H-NMR (CDCl₃): 2.41 (3H, s); 5.74 (1H, s); 7.16 (1H, t, J=8.0 Hz); 7.20-7.26 (1H, m); 7.38-7.40 (1H, m).

### 5-(2-Ethyl-phenyl)-2H-pyrazol-3-yl-amine

### a) 2-Ethyl-benzoic acid methyl ester

2-Ethyl-benzoic acid (3.0 g, 20.0 mmol) was dissolved in MeOH (20 mL) and catalytic quantity of sulfuric acid (1 mL) was added. The mixture was refluxed overnight, after that the solvent was evaporated under reduced pressure; the crude was dissolved in DCM and washed with saturated Na₂CO₃ to basic pH. The organic phase was dried and evaporated under reduced pressure, and the product (2.9 g, yield 88%) was used without further purification

C₁₀H₁₂O₂

¹H-NMR (dmso-d6): 1.12 (3H, t, J=7.2 Hz); 2.86 (2H, q, J=7.2 Hz); 3.81 (3H, s); 7.27-7.34 (2H, m); 7.46-7.51 (1H, m); 7.73-7.75 (1H, m).

### b) 3-(2-Ethyl-phenyl)-3-oxo-propionitrile

The product was prepared according to the general procedure for aminopyrazole synthesis (route A1) from 2-ethyl-benzoic acid methyl ester (2.9 g, 17.6 mmol, 1.0 eq). The crude product was extracted with DCM as a yellow oil and used in the following step without further purification (2.8 g, yield: 92%).

C₁₁H₁₁NO

1H-NMR (dmso-d6): 1.10-1.18 (3H, m); 2.78 (2H, q, J=7.2 Hz); 4.67 (1H, s); 7.23-7.53 (3H, m); 7.73-7.78 (1H, m).

### c) 5-(2-Ethyl-phenyl)-2H-pyrazol-3-yl-amine

The product was prepared according to general procedure for aminopyrazole synthesis (route A2). The crude product was purified through SiO₂ column (20 g) with gradient elution from 100% EtOAc to EtOAc-MeOH 80:20. The title product (1.2 g, yield 40%) was obtained

C₁₁H₁₃N₃

Mass (calculated) [187]; (found) [M+H⁺]=188.

LC Rt=1.58 min, 90% (10 min method)

¹H-NMR (CDCl₃): 1.15 (3H, t, J=7.6 Hz); 2.71 (2H, q, J=7.6 Hz); 5.72 (1H, s); 7.20-7.26 (1H, m); 7.29-7.35 (3H, m).

### 5-(4-Methoxy-phenyl)-4-methyl-2H-pyrazol-3-ylamine

### a) 3-(4-Methoxy-phenyl)-2-methyl-3-oxo-propionitrile

The product was prepared according to the general procedure for aminopyrazole synthesis (route A1) from 4-methoxy-benzoic acid methyl ester (3.0 mL, 18.0 mmol, 1.0 eq), NaH (1.4 g, 36.0 mmol, 2.0 eq) and propionitrile (6.1 mL, 84.9 mmol, 4.7 eq). The crude was purified through Si-column (eluent hexane/ethyl acetate) to give 2.1 g of title product (yield: 62%).

C₁₁H₁₁NO₂

### b) 5-(4-Methoxy-phenyl)-4-methyl-2H-pyrazol-3-ylamine

The product was prepared according to general procedure for aminopyrazole synthesis (route A2). The crude product was washed with basic water and dried, and the title product (1.8 g, yield 80%) was used without further purification

C₁₁H₁₃N₃O

Mass (calculated) [203]; (found) [M+H⁺]=204.

LC Rt=1.34 min, 91% (10 min method)

¹H-NMR (CDCl₃): 2.03 (3H, s); 3.84 (3H, s); 6.96-6.98 (2H, m); 7.37-7.39 (2H, m).

### 4-Methyl-5-(4-trifluoromethyl-phenyl)-2H-pyrazol-3-ylamine

### a) 2-Methyl-3-oxo-3-(4-trifluoromethyl-phenyl)-propionitrile

The product was prepared according to the general procedure for aminopyrazole synthesis (route A1) from 4-trifluoromethyl-benzoic acid methyl ester (3.0 g, 14.7 mmol, 1.0 eq), NaH (1.2 g, 29.4 mmol, 2.0 eq) and propionitrile (4.9 mL, 69.4 mmol, 4.7 eq). The crude product was extracted with DCM and used in the following step without further purification (3.2 g, yield: 96%).

C₁₁H₈F₃NO

### b) 4-Methyl-5-(4-trifluoromethyl-phenyl)-2H-pyrazol-3-ylamine

The product was prepared according to general procedure for aminopyrazole synthesis (route A2). The crude product was washed with basic water and dried, and the title product (2.8 g, yield 84%) was used without further purification

C₁₁H₁₀F₃N₃

Mass (calculated) [241]; (found) [M+H⁺]=242.

LC Rt=2.34 min, 92% (10 min method)

¹H-NMR (CDCl₃): 2.05 (3H, s); 7.56 (2H, d, J=8.4 Hz); 7.64 (2H, d, J=8.4 Hz).

### 5-(4-Cyclopropylmethoxy-2-methyl-phenyl)-2H-pyrazol-3-ylamine

### a) 4-Hydroxy-2-methyl-benzoic acid methyl ester

4-Hydroxy-2-methyl-benzoic acid (4.8 g, 32.0 mmol) was dissolved in MeOH (40 mL) and catalytic quantity of sulfuric acid (1 mL) was added. The mixture was refluxed overnight, after which the solvent was evaporated under reduced pressure; the crude was dissolved in DCM and washed with saturated NaHCO₃ to basic pH. The organic phase was dried and evaporated under reduced pressure, and the product (5.0 g, yield 95%) was used without further purification.

C₉H₁₀O₃

¹H-NMR (dmso-d6): 2.43 (3H, s); 3.72 (3H, s); 6.62-6.64 (2H, m); 7.71-7.73 (1H, m); 10.10 (1H, s).

### b) 4-Cyclopropylmethoxy-2-methyl-benzoic acid methyl ester

4-Hydroxy-2-methyl-benzoic acid methyl ester (1.0 g, 6.0 mmol, 1.0 eq) was dissolved in acetone (14 mL), NaI (0.45 g, 3.0 mmol, 0.5 eq) and K₂CO₃ (1.66 g, 12.0 mmol, 2.0 eq) were added ad the mixture was stirred at room temperature for 20 min. (Bromomethyl)cyclopropane (0.53 mL, 5.4 mmol, 0.9 eq) was added, and the mixture was refluxed for 2 days. The solvent was concentrated under reduced pressure, NaOH 10% was added, and the crude was extracted with DCM and dried. 0.42 g of title product (yield 32%) were recovered and used without further purification.

C₁₃H₁₆O₃

¹H-NMR (CDCl₃): 0.23-0.34 (2H, m); 0.52-0.64 (2H, m); 1.15-1.24 (1H, m); 2.52 (3H, s); 3.75 (2H, d, J=7.2 Hz); 3.77 (3H, s); 6.64-6.66 (1H, m); 7.83-7.85 (2H, m).

### c) 3-(4-Cyclopropylmethoxy-2-methyl-phenyl)-3-oxo-propionitrile

The product was prepared according to the general procedure for aminopyrazole synthesis from 4-cyclopropylmethoxy-2-methyl-benzoic acid methyl ester (route A1bis). 0.54 g of the title product was extracted from water and dried (yield 69%) and used directly for the next step.

C₁₄H₁₅NO₂

### d) 5-(4-Cyclopropylmethoxy-2-methyl-phenyl)-2H-pyrazol-3-ylamine

The product was prepared according to general procedure for aminopyrazole synthesis (route A2). The crude product was purified through SiO₂ column with gradient elution from 100% EtOAc to EtOAc-MeOH 90:10. The title product (206 mg, yield 36%) was obtained.

C₁₄H₁₇N₃O

¹H-NMR (CD₃OD): 0.29-0.36 (2H, m); 0.54-0.63 (2H, m); 1.18-1.28 (1H, m); 2.33 (3H, s); 3.81 (2H, d, J=7.2 Hz); 5.67 (1H, s); 6.74-6.80 (2H, m); 7.25 (1H, d, J=8.8 Hz).

### 5-(3-Chloro-4-cyclopropylmethoxy-phenyl)-2H-pyrazol-3-ylamine

### a) 3-Chloro-4-cyclopropylmethoxy-benzoic acid methyl ester

3-Chloro-4-hydroxy-benzoic acid methyl ester (1.1 g, 6.0 mmol, 1.0 eq) was dissolved in acetone (14 mL), NaI (0.45 g, 3.0 mmol, 0.5 eq) and K₂CO₃ (1.66 g, 12.0 mmol, 2.0 eq) were added ad the mixture was stirred at room temperature for 20 min. (Bromomethyl)cyclopropane (0.53 mL, 5.4 mmol, 0.9 eq) was added, and the mixture was refluxed for 2 days. The solvent was concentrated under reduced pressure, NaOH 10% was added, and the crude was extracted with DCM and dried. The title product (0.88 g, yield 32%) was recovered and used without further purification.

C₁₂H₁₃C₃

¹H-NMR (dmso-d6): 0.33-0.37 (2H, m); 0.55-0.60 (2H, m); 1.25-1.27 (1H, m); 3.80 (3H, s); 3.99 (2H, d, J=7.2 Hz); 7.21 (1H, s, J=8.8 Hz); 7.85-7.91 (2H, m).

### b) 3-(3-Chloro-4-cyclopropylmethoxy-phenyl)-3-oxo-propionitrile

The product was prepared according to the general procedure from 3-Chloro-4-cyclopropylmethoxy-benzoic acid methyl ester (route A1bis). 0.74 g of the title product was extracted from water and dry (yield 81%) and used directly for the next step.

C₁₃H₁₂ClNO₂

### c) 5-(3-Chloro-4-cyclopropylmethoxy-phenyl)-2H-pyrazol-3-ylamine

The product was prepared according to general procedure for aminopyrazole synthesis (route A2). The crude product was purified through SiO₂ column (gradient elution from 100% EtOAc to EtOAc-MeOH 90:10). 521 mg of the title product (yield 67%) were obtained.

C₁₃H₁₄ClN₃O

Mass (calculated) [263]; (found) [M+H⁺]=264.

LC Rt=2.51 min, 90% (10 min method)

¹H-NMR (CD₃OD): 0.25-0.29 (2H, m); 0.52-0.55 (2H, m); 1.10-1.18 (1H, m); 3.81 (2H, d, J=6.8 Hz); 5.74 (1H, s); 6.95-6.99 (1H, m); 7.24-7.30 (2H, m).

### 5-(4-Cyclopropylmethoxy-2-trifluoromethyl-phenyl)-2H-pyrazol-3-ylamine

### a) 4-hydroxy-2-trifluoromethyl-benzoic acid methyl ester

4-hydroxy-2-trifluoromethyl-benzoic acid (5.0 g, 24.0 mmol) was dissolved in MeOH (50 mL) and a catalytic quantity of sulfuric acid was added. The mixture was refluxed overnight, after which the solvent was evaporated under reduced pressure; the crude was dissolved in DCM and washed with saturated NaHCO₃. The organic phase was dried and evaporated under reduced pressure, and the product was used without further purification.

C₉H₇F₃O₃

### b) 4-Cyclopropylmethoxy-2-trifluoromethyl-benzoic acid methyl ester

4-hydroxy-2-trifluoromethyl-benzoic acid methyl ester (1.1 g, 4.8 mmol, 1.0 eq) was dissolved in acetone (14 mL), NaI (0.5 eq) and K₂CO₃ (1.04 g, 2.0 eq) were added and the mixture was stirred at room temperature for 30 min. (Bromomethyl)cyclopropane (0.42 mL, 4.3 mmol, 0.9 eq) was added, and the mixture was refluxed for 2 days. The solvent was concentrated under reduced pressure, NaOH 10% was added, and it was extracted with DCM and dried. The title product (1.21 g, yield 92%) was recovered and used without further purification.

C₁₃H₁₃F₃O₃

### c) 3-(4-Cyclopropylmethoxy-2-trifluoromethyl-phenyl)-3-oxo-propionitrile

The product was prepared according to the general procedure (route A1bis). The mixture was acidified with HCl IM and the organic phase separated and dried, to give 1.2 g of the title product (yield 94%) which was used directly for the next step.

C₁₄H₁₂F₃NO₂

Mass (calculated) [283]; (found) [M+H⁺]=284

LC Rt=3.86 min, 98% (10 min method)

### d) 5-(4-Cyclopropylmethoxy-2-trifluoromethyl-phenyl)-2H-pyrazol-3-ylamine

The product was prepared according to general procedure for aminopyrazole synthesis (route A2). The crude product was purified through SiO₂ column (gradient elution from Ethyl Acetate-cycloexane 1:1 to Ethyl Acetate-MeOH 90:10). 650 mg of the title product (yield 52%) were obtained.

C₁₄H₁₄F₃N₃O

Mass (calculated) [297]; (found) [M+H⁺]=298.

LC Rt=2.78 min, 59% (10 min method)

¹H-NMR (CDCl₃): 032-0.44 (2H, m); 0.64-0.62 (2H, m); 1.22-1.37 (1H, m); 3.80-3.92 (2H, m); 5.78 (1H, s); 7.04-7.07 (1H, m); 7.24-7.26 (1H, m); 7.38-7.40 (1H, m)

### 5-(4-Cyclopropylmethoxy-2,3-difluoro-phenyl)-2H-pyrazol-3-ylamine

### a) 4-hydroxy-2,3-difluoro-benzoic acid methyl ester

4-hydroxy-2,3-difluoro-benzoic acid (2.0 g, 11.5 mmol) was dissolved in MeOH (20 mL) and catalytic quantity of sulfuric acid was added. The mixture was refluxed overnight, after that the solvent was evaporated under reduced pressure; the crude was dissolved in DCM and washed with saturated NaHCO₃. The organic phase was dried and evaporated under reduced pressure, and the product was used without further purification.

C₈H₆F₂O₃

### b) 4-Cyclopropylmethoxy-2,3-difluoro-benzoic acid methyl ester

4-Hydroxy-2,3-difluoro-benzoic acid methyl ester (0.9 g, 4.8 mmol, 1.0 eq) was dissolved in acetone (14 mL), NaI (0.5 eq) and K₂CO₃ (1.03 g, 2.0 eq) were added and the mixture was stirred at room temperature for 30 min. (Bromomethyl)cyclopropane (0.42 mL, 0.9 eq) was added, and the mixture was refluxed for 2 days. The solvent was concentrated under reduced pressure, NaOH 10% was added, and it was extracted with DCM and dried. The title product (0.97 g, yield 84%) was recovered and used without further purification.

C₁₂H₁₂F₂O₃

### c) 3-(4-Cyclopropylmethoxy-2,3-difluoro-phenyl)-3-oxo-propionitrile

The product was prepared according to the general procedure (route A1bis). The mixture was acidified with HCl IM and the organic phase separated and dried, to give 0.79 g of the title product (yield 79%) which was used directly for the next step.

C₁₃H₁₁F₂NO₂

Mass (calculated) [251]; (found) [M+H⁺]=252.

LC Rt=3.53 min, 82% (10 min method)

### d) 5-(4-Cyclopropylmethoxy-2,3-difluoro-phenyl)-2H-pyrazol-3-ylamine

The product was prepared according to general procedure for aminopyrazole synthesis (route A2). The crude product was purified through SiO₂ column (gradient elution from EtOAc-cycloexane 1:1 to EtOAc:MeOH 90:10). 810 mg of the title product (yield 97%) were obtained.

C₁₃H₁₃F₂N₃O

Mass (calculated) [265]; (found) [M+H⁺]=266.

LC Rt=2.59 min, 75% (10 min method)

¹H-NMR (CDCl₃): 032-0.47 (2H, m); 0.64-0.75 (2H, m); 1.19-1.38 (1H, m); 3.67-4.15 (4H, m); 5.95 (1H, s); 6.74-6.88 (1H, m); 7.17-7.26 (1H, m);

### 5-(3,5-Dichloro-4-cyclopropylmethoxy-phenyl)-2H-pyrazol-3ylamine

### a) 3,5-Dichloro-4-Cyclopropylmethoxy-benzoic acid methyl ester

3,5-Dichloro-4-hydroxy-benzoic acid ethyl ester (1.0 g, 4.5 mmol, 1.0 eq) was dissolved in acetone (14 mL), NaI (0.5 eq) and K₂CO₃ (0.98 g, 9.0 mmol, 2.0 eq) were added ad the mixture was stirred at room temperature for 30 min. (Bromomethyl)cyclopropane (0.39 mL, 4.1 mmol, 0.9 eq) was added, and the mixture was refluxed for 2 days. The solvent was concentrated under reduced pressure, NaOH 10% was added, and it was extracted with DCM and dried. The title product (0.98 g, yield 79%) was recovered and used without further purification.

C₁₂H₁₂Cl₂O₃

### b) 3(3,5-Dichloro-4-cyclopropylmethoxy-phenyl)-3-oxo-propionitrile

The product was prepared according to the general procedure (route A1bis). The mixture was acidified with HCl 1M and the organic phase separated and dried, to give 0.91 g of the title product (yield 90%) which was used directly for the next step.

C₁₃H₁₃Cl₂N₃O

Mass (calculated) [283]; (found) [M+H⁺]=284.

LC Rt=4.06 min, 99% (10 min method)

### c) 5-(3,5-Dichloro-4-cyclopropylmethoxy-phenyl)-2H-pyrazol-3ylamine

The product was prepared according to general procedure for aminopyrazole synthesis (route A2). The crude product was purified through SiO₂ column (gradient elution from EtOAc-cycloexane 1:1 to Ethyl Acetate:MeOH 90:10). 750 mg of the title product (yield 79%) were obtained.

C₁₃H₁₃Cl₂N₃O

Mass (calculated) [297]; (found) [M+H⁺]=298.

LC Rt=3.23 min, 93% (10 min method)

¹H-NMR (CDCl₃): 023-0.46 (2H, m); 0.64-0.74 (2H, m); 1.30-1.48 (1H, m); 3.60-4.04 (4H, m); 5.86 (1H, s); 7.48 (2H, s)

### 5-(4-Cyclopropylmethoxy-3-methoxy-phenyl)-2H-pyrazol-3-ylamine

### a) 4-Cyclopropylmethoxy-3-methoxy-benzoic acid methyl ester

4-hydroxy-3-methoxy-benzoic acid methyl ester (1.0 g, 5.5 mmol, 1.0 eq) was dissolved in acetone (14 mL), NaI (0.5 eq) and K₂CO₃ (1.0 g, 2.0 eq) were added and the mixture was stirred at room temperature for 30 min. (Bromomethyl)cyclopropane (0.53 mL, 0.9 eq) was added, and the mixture was refluxed for 2 days. The solvent was concentrated under reduced pressure, NaOH 10% was added, and it was extracted with DCM and dried. The title product (1.21 g, yield 93%) was recovered and used without further purification.

C₁₃H₁₆O₄

### b) 3(4-Cyclopropylmethoxy-3-methoxy-phenyl)-3-oxo-propionitrile

The product was prepared according to the general procedure (route A1bis). The mixture was acidified with HCl IM and the organic phase separated and dried, to give 1.24 g of the title product (yield 99%) which was used directly for the next step.

C₁₄H₁₅NO₃

Mass (calculated) [245]; (found) [M+H⁺]=246.

LC Rt=3.03 min, 100% (10 min method)

### c) 5-(4-Cyclopropylmethoxy-3-methoxy-phenyl)-2H-pyrazol-3-ylamine

The product was prepared according to general procedure for aminopyrazole synthesis (route A2). The crude product was purified through SiO₂ column (gradient elution from EtOAc-cycloexane 1:1 to Ethyl Acetate:MeOH 90:10). 220 mg of the title product (yield 50%) were obtained.

C₁₄H₁₇N₃O₂

Mass (calculated) [259]; (found) [M+H⁺]=260.

LC Rt=1.86 min, 93% (10 min method)

¹H-NMR (CDCl₃): 027-0.43 (2H, m); 0.56-0.72 (2H, m); 1.23-1.40 (1H, m); 348 (2H, m); 3.87 (3H, s); 3.98 (2H, br s); 5.82 (1H, s); 6.85-6.89 (1H, m); 7.05-7.10 (2H, m);

### 3-Amino-5-(3-fluoro-phenyl)-pyrazole-1-carboxylic acid tert-butyl ester

3-Amino-5-(3-fluoro-phenyl)-pyrazole (5.0 g, 28.0 mmol, 1.0 eq) and KOH 4.5 M (50 mL, 226 mmol, 8 eq) were dissolved in DCM (200 mL), and di-tert-butyl dicarbonate (6.5 g, 30.0 mmol, 1.1 eq) was added; the mixture was stirred at room temperature until complete conversion was observed by LC-MS analysis. The organic phase was washed with saturated brine and evaporated; the crude was crystallized with MeOH, to give 7.4 g of title product (yield 95%).

C₁₄H₁₆FN₃O₂

¹H-NMR (dmso-d6): 1.57 (9H, s), 5.80 (1H, s), 6.43 (2H, br s), 7.16-7.21 (1H, m), 7.41-7.47 (1H, m); 7.50-7.54 (1H, m); 7.58-7.60 (1H, m).

### 3-Amino-5-o-tolyl-pyrazole-1-carboxylic acid tert-butyl ester

3-Amino-5-o-tolyl-pyrazole (0.5 g, 2.89 mmol, 1.0 eq) and KOH 4.5 M (5.1 mL, 23.1 mmol, 8.0 eq) were dissolved in DCM (20 mL), and Di-tert-butyl dicarbonate (0.66 g, 3.0 mmol, 1.1 eq) was added; the mixture was stirred at room temperature until complete conversion was observed by LC-MS analysis. The organic phase was washed with saturated brine and evaporated, to give 0.6 g of title product (yield 76%).

C₁₅H₁₉N₃O₂

Mass (calculated) [273]; (found) [M+H⁺]=274.

LC Rt=2.34 min, 96% (5 min method)

### 3-Amino-5-(4-trifluoromethyl-phenyl)-pyrazole-1-carboxylic acid tert-butyl ester

3-Amino-5-(4-trifluoromethyl-phenyl)-pyrazole (2.0 g, 8.8 mmol, 1.0 eq) and KOH 4.5 M (15.7 mL, 70.5 mmol, 8.0 eq) were dissolved in DCM (70 mL), and di-tert-butyl dicarbonate (2.02 g, 9.2 mmol, 1.1 eq) was added; the mixture was stirred at room temperature until complete conversion was observed by LC-MS analysis. The organic phase was washed with saturated brine and evaporated; the crude was crystallized with CH₃CN, to give 1.9 g of title product (yield 69%).

Mass (calculated) [327]; (found) [M+H⁺]=328.

LC Rt=2.59 min, 100% (5 min method)

¹H-NMR (dmso-d6): 1.57 (9H, s), 5.83 (1H, s), 6.46 (2H, s), 7.74 (2H, d, J=8.4 Hz), 7.95 (2H, d, J=8.8 Hz)

### 5-Pyridin-2-yl-2H-pyrazol-3-ylamine

### a) Oxo-pyridin-2-yl-acetonitrile

The product was prepared according to the general procedure for aminopyrazole synthesis (route A1) from pyridine-2-carboxylic acid methyl ester (3.0 g, 21.9 mmol, 1.0 eq). The crude was precipitated from HCl to give the title product as a solid (2.2 g, yield: 69%) which was used directly for the next step.

C₈H₆N₂O

### b) 5-Pyridin-2-yl-2H-pyrazol-3-ylamine

The product was prepared according to general procedure for aminopyrazole synthesis (route A2). The crude product was dissolved in EtOAc, washed with NaHCO₃, dried and evaporated. NMR analysis showed that a major portion of the crude mixture was still in the opened form: the mixture was then dissolved in CH₃COOH and heated at 80° C. overnight, to allow for ring closure of the opened form. The product was then recovered as the acylated form, which was de-acylated stirring with HCl 6N at 60° C. overnight obtaining the title product (0.816 g, yield 60%).

C₈H₈N₄

¹H-NMR (dmso-d6): 4.81 (2H, bs), 5.92 (1H, s), 7.21-7.24 (1H, m), 7.76 (2H, d), 8.51 (1H, d), 11.96 (1H, bs)

### 5-(3-Difluoromethoxy-phenyl)-2H-pyrazol-3-ylamine

### a) 3-Difluoromethoxy-benzoic acid methyl ester

Difluoromethoxy-benzoic acid (2.0 g, 10.6 mmol, 1.0 eq) was dissolved in MeOH (15 mL) and a catalytic quantity of sulfuric acid was added. The mixture was refluxed overnight, after which the solvent was evaporated under reduced pressure; the crude was dissolved in DCM and washed with saturated NaHCO₃ to basic pH. The organic phase was dried and evaporated under reduced pressure, and the title product was used without further purification (1.9 g, yield 90%).

C₉H₈F₂O₃

¹H-NMR (dmso-d6): 3.86 (3H, s), 7.33 (1H, t, J=73.6 Hz), 7.46-7.50 (1H, m), 7.59 (1H, t, J=8.0 Hz), 7.67 (1H, s); 7.82 (1H, d, J=7.6 Hz).

### b) 3-(3-Difluoromethoxy-phenyl)-3-oxo-propionitrile

The product was prepared according to the general procedure for aminopyrazole synthesis (route A1 bis) from 3-difluoromethoxy-benzoic acid methyl ester (1.5 g, 7.4 mmol, 1.0 eq). The crude was precipitated by addition of aqueous HCl to give the product which was used directly for the next step.

C₁₀H₇F₂NO₂

### c) 5-(3-Difluoromethoxy-phenyl)-2H-pyrazol-3-ylamine

The product was prepared according to general procedure for aminopyrazole synthesis (route A2). The crude product was purified through Si-column with gradient elution from 100% EtOAc to EtOAc-MeOH 90:10. 1.45 g of title product (yield 87%) was obtained.

C₁₀H₉F₂N₃O

¹H-NMR (dmso-d6): 4.89 (2H, br s), 5.75 (1H, s), 7.02 (1H, d), 7.25 (1H, t, J=74.0 Hz), 7.36-7.42 (2H, m), 7.48-7.50 (1H, d), 11.76 (1H, br s)

### 5-Pyrazolo[1,5-a]pyridin-3-yl-2H-pyrazol-3-ylamine

### a) 3-Oxo-3-pyrazolo[1,5-a]pyridin-3-yl-propionitrile

To a solution of dry acetonitrile in toluene (0.66 mL, 13 mmol, 5 eq) cooled down to −78° C. under nitrogen, a solution of n-butyllithium in n-hexane (5.2 mL, 13 mmol, 5 eq) was added dropwise. The mixture was left stirring at −78° C. for 20 minutes and then a solution of pyrazolo[1,5-a]pyridine-3-carboxylic acid methyl ester (0.46 g, 2.6 mmol, 1 eq, prepared according to the reported procedure (Anderson et al. Journal of Heterocyclic Chemistry 1981, 18, 1149-1152) in toluene was added and the reaction allowed to reach room temperature. Upon reaction completion, after about 20 minutes, the mixture was cooled down to 0° C. and HCl 2N was added to pH 2. The organic phase was recovered, dried over Na₂SO₄ and concentrated under reduced pressure, affording the title product which was used without further purification in the following step.

C₁₀H₇N₃O

### b) 5-Pyrazolo[1,5-a]pyridin-3-yl-2H-pyrazol-3-ylamine

To a solution of the 3-oxo-3-pyrazolo[1,5-a]pyridin-3-yl-propionitrile (0.66 g, 3.6 mmol), in absolute EtOH (25 mL) hydrazine monohydrate (0.44 mL, 9.0 mmol) was added and the reaction was heated at reflux for 18 hours. The reaction mixture was allowed to cool to room temperature and the solvent was evaporated under reduced pressure. The residue was dissolved in DCM and washed with water.

The organic phase was concentrated under reduced pressure to give a crude product that was purified by SiO₂ column (DCM to DCM:MeOH 95:5 to 85:15 gradient), yielding the title compound in 41% Yield (0.29 g, 1.48 mmol).

C₁₀H₉N₅

¹H-NMR (dmso-d6): 8.68 (s, 1H); 8.21 (s, 1H); 7.92 (s, 1H); 7.28 (s, 1H); 6.90 (s, 1H); 5.75 (s, 1H); 5.10 (s, 2H).

Mass (calculated) [199]; (found) [M+H⁺]=200.

LC Rt=0.86 min, 92% (5 min method).

### Example 1

### 5-(4-Acetyl-[1,4]diazepan-1-yl)-pentanoic acid [5-(4-methoxy-phenyl)-1H-pyrazol-3-yl]-amide

### i) 5-Bromo-pentanoic acid [5-(4-methoxy-phenyl)-1H-pyrazol-3-yl]-amide

A solution of 5-bromovaleryl chloride (2.1 mL, 15.7 mmol, 1 eq) in dry DMA (35 mL) was cooled to −10° C. (ice/water bath) under N₂; a solution of 5-(4-methoxy-phenyl)-1H-pyrazol-3-ylamine (3.0 g, 15.7 mmol, 1 equiv.) and diisopropylethylamine (2.74 mL, 15.7 mmol, 1 equiv.) in dry DMA (15 mL) was added over 30 min. After 2 hrs at −10° C., LC-MS shows completion of the reaction which was quenched by addition of H₂O (ca. 50 mL). The solid which precipitates was filtered and washed with Et₂O, to give 4.68 g of 5-bromo-pentanoic acid [5-(4-methoxy-phenyl)-1H-pyrazol-3-yl]-amide as a white powder (13.3 mmol, 85% yield).

mp=149.5-151.5° C.

C₁₅H₁₈BrN₃O₂ Mass (calculated) [352.23]; (found) [M+H⁺]=352.09/354.10

LC Rt=2.07, 95% (5 min method)

¹H-NMR (400 MHz, DMSO-d6): δ 1.69-1.63 (2H, m); 1.81-1.75 (2H, m); 2.29 (2H, t); 3.52 (2H, t); 3.75 (3H, s); 6.75 (1H, bs); 6.96 (2H, d); 7.6 (2H, d); 10.28 (1H, s); 12.57 (1H, s)

### ii) 5-(4-Acetyl-[1,4]diazepan-1-yl)-pentanoic acid [5-(4-methoxy-phenyl)-1H-pyrazol-3-yl]-amide

To 750 mg (1.96 mmol) of 5-bromo-pentanoic acid [5-(4-methoxy-phenyl)-2H-pyrazol-3-yl]-amide in 7 mL of DMA, N-acetyl-diazepine (278 mg, 1.96 mmol) and NaI (240 mg, 1.96 mmol) were added and the reaction heated at 60° C. for 18 hours. Upon complete conversion (as monitored by LC-MS) the mixture was diluted with 20 mL of DCM and washed with water. The organic phase was concentrated under reduced pressure to afford a residue which was purified with a SiO₂ column (10 g) eluting with a gradient from DCM to MeOH 90:10. The title compound (380 mg) was recovered pure (yield 46%).

C₂₂H₃₁N₅O₃ Mass (calculated) [413]; (found) [M+H⁺]=414

LC Rt=1.91, 100% (10 min method)

¹H-NMR (400 MHz, DMSO-d6): δ 1.53-1.75 (4H, m), 1.90-2.15 (5H, m), 2.28-2.42 (2H, m), 2.90-3.26 (3H, m), 3.34-3.58 (3H, m), 3.71-3.88 (7H, m)

### Example 2

### 5-(4-acetyl-1,4-diazepan-1-yl)-N-(5-(4-methoxyphenyl)-1H-pyrazol-3-yl)pentanamide (mono hydrochloride salt)

To a solution of 5-(4-methoxyphenyl)-1H-pyrazol-3-ylamine (12 g, 62.8 mmol) and N,N-diisopropylethylamine (10.96 mL, 62.8 mmol) in dry N,N-dimethylformamide (150 mL) at −10° C. was added a solution of 5-bromovaleryl chloride (8.4 mL, 62.8 mmol) in dry N,N-dimethylformamide (50 mL) slowly (˜40 min) and the reaction mixture was allowed to stir at −10 to 0° C. for 8 hrs. Sodium iodide (9.44 g, 62.8 mmol) was added at 0° C. and followed by N-acetylhomopiperazine (8.24 mL, 62.8 mmol) and N,N-diisopropylethylamine (10.96 mL, 62.8 mmol) and the reaction mixture was allowed to stir at 50° C. for 18 hrs. The solvent was removed in vacuo. The residue was dissolved in methylene chloride (500 mL) and saturated aqueous sodium bicarbonate (500 mL) and the mixture was stirred at room temperature for 30 minutes. The organic layer was separated, dried over sodium sulfate, and the solvent was removed in vacuo to provide 25.8 g (99%) of 5-(4-acetyl-1,4-diazepan-1-yl)-N-(5-(4-methoxyphenyl)-1H-pyrazol-3-yl)pentanamide as a thick light yellow oil (crude).

Then to a solution of the crude 5-(4-acetyl-1,4-diazepan-1-yl)-N-(5-(4-methoxyphenyl)-1H-pyrazol-3-yl)pentanamide (as a free base) in methylene chloride (270 mL) at room temperature was added hydrogen chloride (65 mL, 1.0 M in ethyl ether) slowly. The resulting suspension was allowed to stir at room temperature for 1 hour. The solvent was removed in vacuo to afford 33 g as a yellow foam, mono hydrochloride salt. The foam was dissolved in solvents (330 mL, acetonitrile:methanol=33:1) at 60-70° C. and a crystal seed was added. The mixture was slowly cooled down to the room temperature and allowed to stir at room temperature for 15 hours. The resulting precipitate was filtered and dried to give 20.5 g (72%) of the title compound as a white crystal, mono hydrochloride salt. MS [M−H]⁻ m/z 412.3; mp. 132-133° C. ¹H NMR (400 MHz, DMSO-d₆) δ 10.6-10.8 (br, 1H), 10.45 (s, 1H), 7.64 (d, J=8 Hz, 2H), 7.00 (d, J=8 Hz, 2H), 6.74 (s, H), 4.00 (m, 1H), 3.77 (s, 3H), 3.4-3.6 (m, 6H), 2.9˜3.0 (m, 5H), 2.34 (m, 2H), 2.0 (s, 3H), 1.65-1.70 (m, 2H), 1.55-1.65 (m, 2H).

### Example 3

### 5-(4-acetyl-1,4-diazepan-1-yl)-N-(5-(4-methoxyphenyl)-1H-pyrazol-3-yl)pentanamide (mono hydrochloride salt)

### i) 3-(4-methoxyphenyl)-3-oxopropanenitrile

A solution of methyl p-anisate in acetonitrile was cooled to −10° C. Lithium bis(trimethylsilyl)amide (1 M in THF) was added dropwise over a minimum of 3 hr. The mixture was held at −10 to 0° C. until reaction completion. The reaction mixture was quenched with water and the pH adjusted to 3-4 with conc HCl. The mixture was stirred for 1 hr. The product was isolated by filtration, washed with water and dried in a vacuum oven. The yield was 73%.

### ii) 5-(4-methoxyphenyl)-1H-pyrazol-3-amine

A suspension of 3-(4-methoxyphenyl)-3-oxopropanenitrile in ethanol was heated to 60° C. Hydrazine hydrate was added dropwise over a minimum of 30 min at 60° C. The resulting solution was held at 60° C. until reaction completion, generally 15-18 hr. The reaction mixture was quenched with water. Ethanol was removed by distillation to about 5 volumes. The product was isolated by filtration, washed with water and dried in a vacuum oven. The yield was 88-95%.

### iii) 5-bromo-N-(5-(4-methoxyphenyl)-1H-pyrazol-3-yl)pentanamide

A solution of 5-(4-methoxyphenyl)-1H-pyrazol-3-amine and diisopropylethylamine in 10 volumes of a 9:1 mixture of acetonitrile:DMF was cooled to −10° C. 5-Bromovaleryl chloride was added dropwise over a minimum of 3 hr at −10° C. The resulting solution was held at −10° C. until reaction completion, generally 2 hr. The reaction mixture was quenched with water. The product was isolated by filtration, washed with water, TBME and suction dried. The product-wet cake was purified by re-slurrying in TBME at 35° C. for a minimum of 2 hr. The yield was 70-80%.

### iv) 5-(4-acetyl-1,4-diazepan-1-yl)-N-(5-(4-methoxyphenyl)-1H-pyrazol-3-yl)pentanamide hydrochloride

Bromopyrazole is mixed with K₂CO₃ and KI in 10 volumes of acetone at room temperature and N-acetylhomopiperazine was added over 1 hr. The reaction mixture was stirred until the reaction was complete. The mixture was filtered, removing the inorganics, washed with acetone and distilled to 2 volumes. The freebase was extracted into methyl THF/EtOH and washed with NaCl and NaHCO₃. The solvent was replaced with EtOH, a strength of the solution was determined, and 0.93 eq of HCl based on the available freebase was added to a mixture of acetone, ethanol and water. Careful monitoring of the pH yielded crystalline product in a 70% overall yield and the desired form 1.

### Example 4

### 5-(4-acetyl-1,4-diazepan-1-yl)-N-(5-(4-methoxyphenyl)-1H-pyrazol-3-yl)pentanamide (mono hydrochloride salt)

### i) 5-(4-methoxy-phenyl)-1H-pyrazol-3-ylamine

The intermediate 5-(4-methoxy-phenyl)-1H-pyrazol-3-ylamine is commercially available from Sigma-Alrich (USA), but can be made using the following general procedure:

Aryl β-Ketonitrile Synthesis

To a solution of an aromatic ester (6.5 mmol) in dry toluene (6 mL), under N₂, NaH (50-60% dispersion in mineral oil, 624 mg, 13 mmol) was carefully added. The mixture was heated at 80° C. and then dry CH₃CN was added dropwise (1.6 mL, 30.8 mmol). The reaction was heated for 18 h and generally the product precipitated from the reaction mixture as a salt. The reaction was allowed to cool to room temperature and the solid formed was filtered and then dissolved in water. The solution was acidified with 2 N HCl solution, and upon reaching a pH between 2-4, the product precipitated and was filtered. If no precipitation occurred, the product was extracted with DCM. After aqueous workup, the products were generally pure enough to be used in the next step without further purification. The isolated yield was generally 40-80%.

Aryl Aminopyrazole Synthesis

To a solution of β-ketonitrile (7.5 mmol) in absolute EtOH (15 mL), hydrazine monohydrate (0.44 mL, 9.0 mmol) was added and the reaction was heated at reflux for 18 hrs. The reaction mixture was allowed to cool to room temperature and the solvent was evaporated under reduced pressure. The residue was dissolved in 20 mL of DCM and washed with water. The organic phase was concentrated to give a crude product that was purified by SiO₂ column or by precipitation from Et₂O. For example, the 2-methoxy derivative was purified by SiO₂ chromatography, eluting with a DCM/MeOH gradient (from 100% DCM to 90/10 DCM/MeOH); the 3-methoxy derivative was triturated with Et₂O. Yields were generally 65-90%.

### ii) 5-bromo-pentanoic acid [5-(4-methoxy-phenyl)-1H-pyrazol-3-yl]amide

A solution of 5-bromovaleryl chloride (2.1 mL, 15.7 mmol) in dry dimethylacetamide (DMA) (35 mL) was cooled to −10° C. (ice water bath) under N₂; a solution of 5-(4-methoxy-phenyl)-1H-pyrazol-3-ylamine (3.0 g, 15.7 mmol) and diisopropylethylamine (2.74 mL, 15.7 mmol) in dry DMA (15 mL) was added over 30 min. After two hours at −10° C., LCMS shows completion of the reaction (acylation on the pyrazole ring was also detected). The reaction was quenched by addition of H₂O (ca. 50 mL), and the thick white precipitate formed upon addition of water was recovered by filtration. When the reaction was allowed to reach room temperature before quenching, a putative exchange of Br with Cl caused reactivity problems in subsequent steps. Washing with Et₂O (3×10 mL) efficiently removed the byproduct (acylation on pyrazole ring). 4.68 g of the title compound was obtained as a white powder (13.3 mmol, 85% yield). Mp=149.5-151.5° C.

### iii) 5-(4-acetyl-[1,4]diazepan-1-yl)-pentanoic acid [5-(4-methoxy-phenyl)-1H-pyrazol-3-yl]-amide

5-bromo-pentanoic acid [5-(4-methoxy-phenyl)-1H-pyrazol-3-yl]amide (1.5 g, 4.26 mmol) was dissolved in DMF (15 mL), and sodium iodide (0.64 g, 4.26 mmol) was added followed by N-acetylhomopiperazine (0.56 mL, 4.26 mmol) and diisopropylethylamine (0.74 mL, 4.26 mmol). The reaction was stirred under N₂ at 50° C. for 18 hrs. Upon reaction completion (as monitored by LCMS), the solvent was removed at reduced pressure and the resulting oily residue was dissolved in DCM (20 mL), washed with sat. Na₂CO₃ (2×20 mL) and sat. NaCl (2×20 mL), and dried over Na₂SO₄. Upon solvent removal, 1.7 g of crude product as a thick oil were obtained. The product was purified by SiO₂ chromatography (10 g cartridge-flash SI II from IST) employing DCM and DCM:MeOH 9:1 to yield 0.92 g of pure product and 0.52 g of less pure product. A second purification of the impure fractions using a 5 g SiO₂ cartridge was performed using the same eluent. Overall, 1.09 g of 5-(4-acetyl-[1,4]diazepan-1-yl)-pentanoic acid [5-(4-methoxy-phenyl)-1H-pyrazol-3-yl]-amide were obtained (2.64 mmol, 62% yield) as a thick light yellow oil. MS (ES+): 414.26 (M+H)⁺.

### iv) 5-(4-acetyl-1,4-diazepan-1-yl)-N-(5-(4-methoxyphenyl)-1H-pyrazol-3-yl)pentanamide (mono hydrochloride salt)

5-(4-acetyl-[1,4]diazepan-1-yl)-pentanoic acid [5-(4-methoxy-phenyl)-1H-pyrazol-3-yl]-amide (1.05 g, 2.54 mmol) was dissolved in a minimum amount of DCM (5 mL) and cooled to 0° C. HCl (2.0 M in Et₂₀, 1.4 mL, 2.89 mmol) was added and the mixture stirred at rt until precipitation of the salt was complete (about 10 min.). The solid was filtered, washed with Et₂O several times, and dried in a dessicator to yield 1.09 g of the hydrochloride salt (2.42 mmol, 95% yield). Melting point was not determined due to the extreme hygroscopicity of the sample. MS (ES+): 414.26 (M+H)⁺.

### Example 5

### 5-(4-acetyl-1,4-diazepan-1-yl)-N-(5-(4-methoxyphenyl)-1H-pyrazol-3-yl)pentanamide (mono hydrochloride salt)

### i) 5-(4-acetyl-[1,4]diazepan-1-yl)-N-[5-(4-methoxy-phenyl)-1H-pyrazol-3-yl]-pentanamide

To a cylindrical, jacketed 3 L reactor equipped with nitrogen inerting, agitator, condenser/distillation head, and temperature control, 5-bromo-pentanoic acid [5-(4-methoxy-phenyl)-1H-pyrazol-3-yl]amide (0.15 kg, 0.426 mol), potassium carbonate (0.059 kg, 0.426 mol), potassium iodide (0.071 kg, 0.426 mol), and acetone (1.18 kg, 1.5 L) were added (at 20° C.) to form a white mixture. The mixture was stirred (235 rpm) at 25-30° C. for a minimum of 15 min. N-acetylhomopiperazine (0.062 kg, 0.057 L, 0.434 mol) was added via addition funnel to the reactor over a minimum of 45 min., maintaining the temperature in the range of 25-30° C. The addition funnel was rinsed with 0.05 L acetone. A white mixture persisted. The mixture was stirred (235 rpm) in the range of 25-30° C. for a minimum of 16 h, forming a white/yellow mixture. The reaction progress was monitored by HPLC and was considered complete when there was ≦2% of the starting material (bromopyrazole) and ≦2% of the iodopyrazole present.

The reactor contents were cooled to 5-15° C. over a minimum of 15 min with agitation (295 rpm) to form a white/yellow mixture that was then stirred for a minimum of 1 h. To remove inorganics, the mixture was then filtered on a Buchner funnel with filter paper using house vacuum for 1.5 min. The cake was washed twice with acetone (total of 0.24 kg, 0.30 L) at 5-15° C. The wash was combined with the mother liquor from the prior filtration and used to rinse the reactor. The filtrate was concentrated to a volume of approximately 0.45 L to form a clear solution.

### ii) Aqueous Workup

To a reactor containing the material from step i, 1.5 L of a freshly made homogeneous solution of methyl THF (1.22 kg, 1.42 L) and ethanol (0.059 kg, 0.075 L; 99.5% ethanol, 0.5% toluene) was added at 25° C., forming a hazy solution. To this, 0.45 L of a 5% solution of sodium chloride (0.022 kg) in water (0.43 L) was added at 25° C. The resulting mixture was heated with stirring to 30-35° C. over a minimum of 15 min., forming a clear biphasic solution. The agitation was stopped to allow the layers to settle, the product being in the upper layer. The layers were separated, keeping any emulsion in the upper organic layer. The organic layer was retained. A homogeneous 5% solution of sodium bicarbonate (0.03 kg) in water (0.57 L) at 25° C. was used to wash organic layer, stirring for a minimum of 5 min. at 10-15° C. The agitation was stopped to allow the layers to settle, the product being in the upper layer. The layers were separated, keeping any emulsion in the upper organic layer. The organic layer was retained and concentrated to a volume of 0.35 L, forming a hazy solution. The mixture was chased with ethanol to remove residual water.

### iii) 5-(4-acetyl-[1,4]diazepan-1-yl)-N-[5-(4-methoxy-phenyl)-1H-pyrazol-3-yl]-pentanamide HCl

To a reactor containing the material from step ii, 0.47 kg (0.60 L) of acetone was added. The resulting mixture was heated with stirring to 25-30° C. over a minimum of 10 min., forming a hazy solution. The contents of the reactor were clarified through a polypropylene pad into a tared 2 L suction flask using vacuum, maintaining the contents of the reactor at 25-30° C. Suction was maintained until filtration stopped. The reactor and filter pad were rinsed with acetone (0.05 L) at 20-25° C. An accurate strength of the free base was determined. The filtrates from the suction flask were transferred to the reactor and rinsed using acetone (0.05 L). A solution of 5% HCl (0.042 kg, 0.036 L) in acetone (0.174 L) and alcohol solution (0.0174 L of ethanol:acetone (91:9) v/v) was prepared and stirred until homogeneous at 10° C. To the reactor, 0.05 L of water was added to form a clear solution. One third of the 5% HCl solution (0.076 L) was added to the reactor over a minimum of 20 min., maintaining the temperature in the range of 20-25° C. A second third of the 5% HCl solution (0.076 L) was then added to the reactor over a minimum of 20 min., maintaining the temperature in the range of 20-25° C. The contents of the reactor were seeded with 75 mg of 5-(4-acetyl-[1,4]diazepan-1-yl)-N-[5-(4-methoxy-phenyl)-1H-pyrazol-3-yl]-pentanamide HCl (e.g., Form 1), followed by the addition of the last third of 5% HCl solution (0.076 L) over a minimum of 20 min., maintaining the temperature in the range of 20-25° C. Another 0.08 equiv. of the 5% HCl solution (0.023 L) was then added to the reactor over a minimum of 30 min., maintaining the temperature in the range of 20-25° C. Judicious monitoring of pH was performed to attain the desired pH range of 5.2-5.8. Based on the strength calculation, 0.85 equiv. of acid was added over 1 hr and the remaining acid was added over a minimum of 30 min. with careful monitoring of pH.

The mixture was stirred at 20-25° C. for a minimum of 1 hr., forming a thin suspension. Acetone (0.6 L) was added over a minimum of 60 min., maintaining the temperature in the range of 20-25° C. The mixture was stirred at 20-25° C. for a minimum of 60 min. Acetone (1.5 L) was added to the reactor over a minimum of 3 hr., maintaining the temperature in the range of 20-25° C., forming a thick suspension. The mixture was then stirred at 20-25° C. for a minimum of 12 h. Crystallization was considered complete when there was ≦20% of the product present in the mother liquor. Longer stirring was employed if crystallization was not complete.

The mixture was then filtered on a Buchner funnel (polypropylene pad) using house vacuum. A solution of water (0.009 L), acetone (0.23 L) and 0.06 L alcohol (ethanol:acetone (91:9) v/v) was stirred until homogeneous (20% ethanol, 3% water, 77% acetone overall). This solution was used to wash the filter cake twice (0.15 L×2). A solution of water (0.009 L), acetone (0.171 L) and 0.12 L alcohol (ethanol:acetone (91:9) v/v) was stirred until homogeneous (40% ethanol, 3% water, 57% acetone overall). This solution was used to wash the filter cake (0.30 L). The wet cake was subjected to suction under nitrogen using house vacuum and held for 30 min. after dripping stopped. Product purity was checked by HPLC and additional washing was performed if total impurities were not ≦2%. Product was oven dried in a vacuum oven with nitrogen bleed at 38-45° C., maintaining vacuum at 20 torr for a minimum of 12 h until loss on drying of less than 1% was obtained. Following drying, 0.119 kg of the title compound was obtained in 62% yield (67% adjusted for aliquots removed during process; 60% when corrected for strength or purity). Melting point=185° C.; crystal form=form 1; particle size=D90<89.4 um, D50<19.2 um.

### Example 6

### 5-(4-Acetyl-[1,4]diazepan-1-yl)-pentanoic acid [5-(4-methoxy-phenyl)-1H-pyrazol-3-yl]-amide (hydrochloride salt) crystal forms

The present Example describes the preparation of the hydrochloride salt form of 5-(4-Acetyl-[1,4]diazepan-1-yl)-pentanoic acid [5-(4-methoxy-phenyl)-2H-pyrazol-3-yl]-amide. The hydrochloric acid salt form readily adopted a solid form. Indeed, at least four different crystalline forms (i.e., polymorphs) were observed for the hydrochloric acid salt form (see below).

| Counter Ion Used | Solid Obtained | Melting Onset | Hygroscopicity |
|---|---|---|---|
| Hydrochloric acid | Crystalline solid | 185° C. | No |
| | | 165° C. | Somewhat |
| | | 125° C. | Yes |
| | | 125° C. | Not Measured |
| | | three peaks: | Yes |
| | | about 100 | |
| | | about 180; and | |
| | | about 200° C. | |

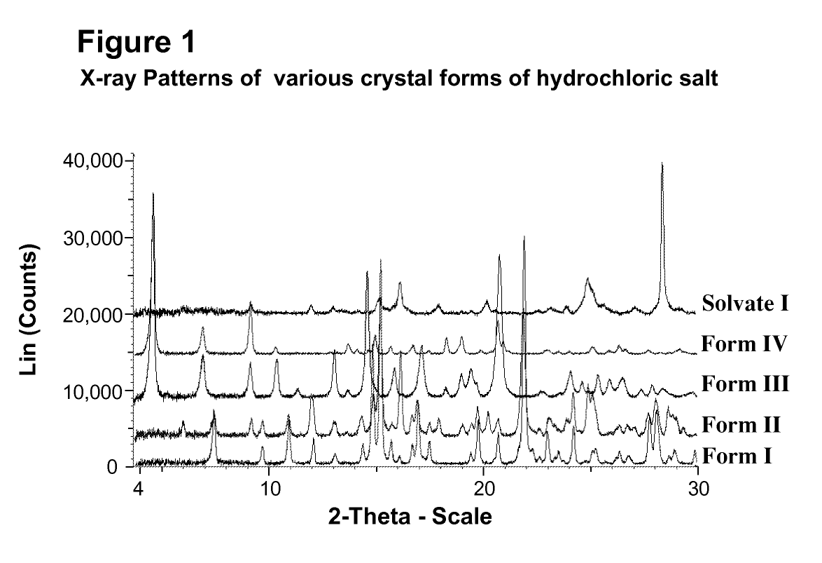

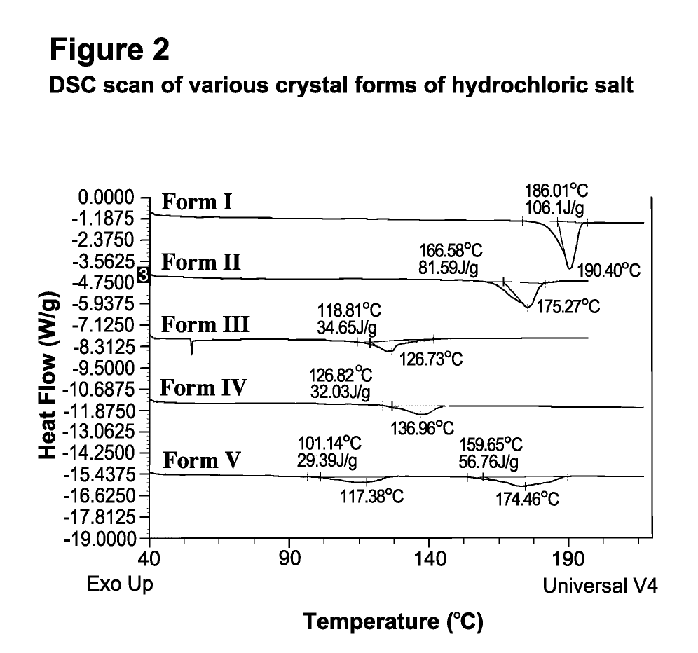

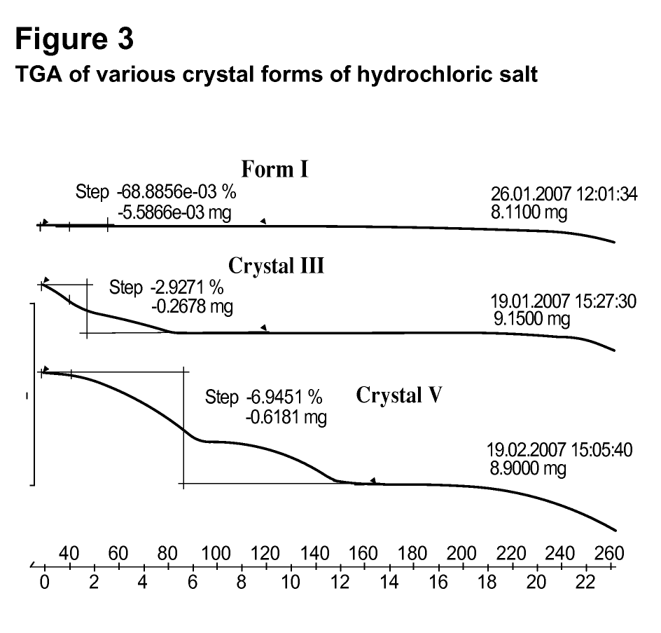

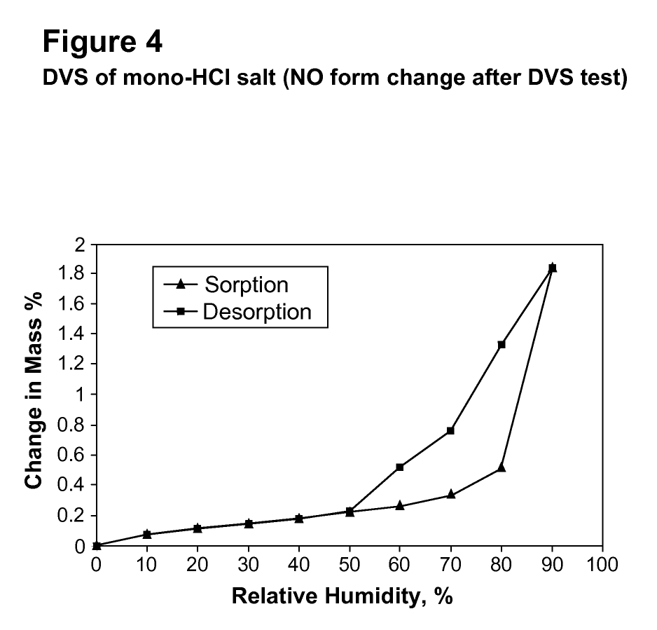

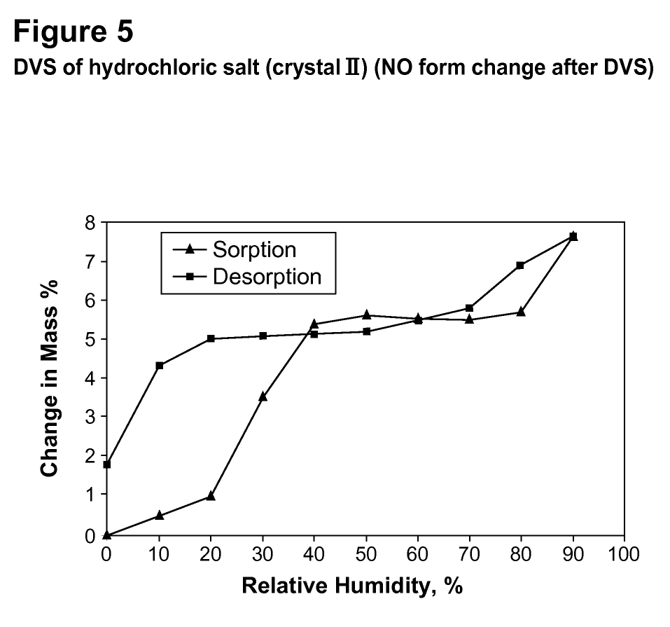

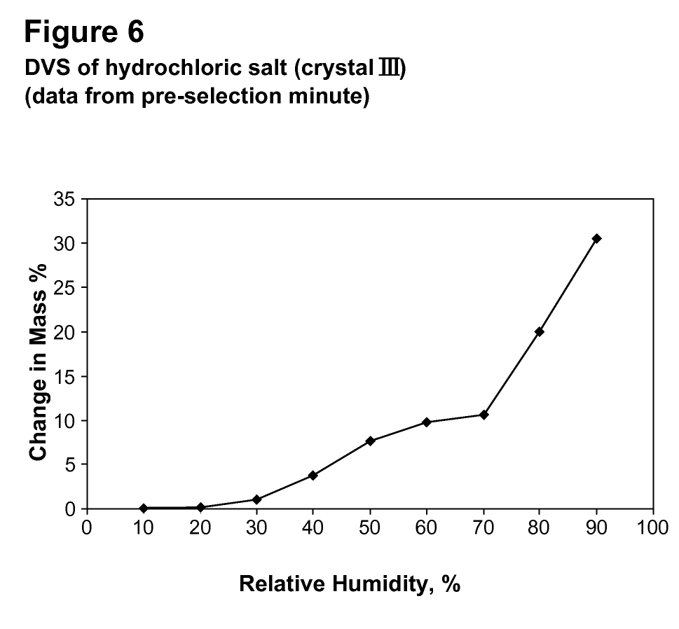

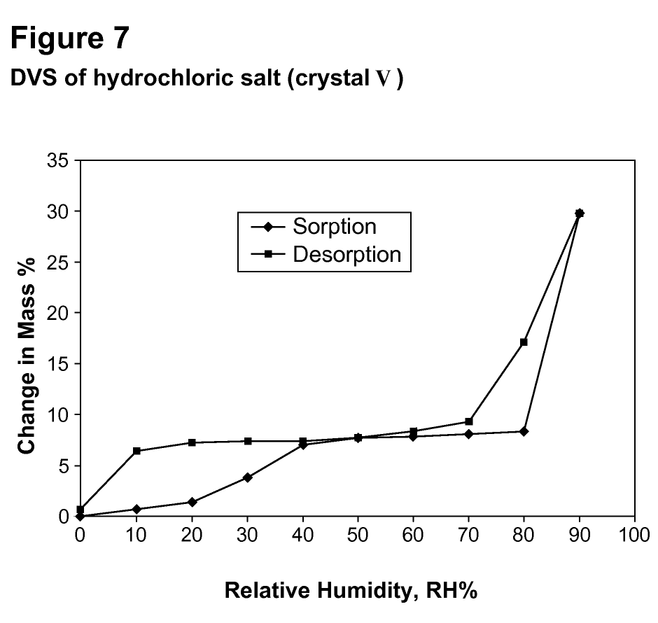

Differential scanning calorimetry data were collected for each solid form achieved using a DSC (TA instruments, model Q1000) under the following parameters: 50 mL/min purge gas (N2); scan range 40 to 200° C., scan rate 10° C./min. Thermogravimetric analysis data were collected using a TGA instruments (Mettler Toledo, model TGA/SDTA 851e) under the following parameters: 40 ml/min purge gas (N2); scan range 30 to 250° C., scan rate 10° C./min. X-ray data were acquired using an X-ray powder diffractometer (Bruker-axs, model D8 advance) having the following parameters: voltage 40 kV, current 40.0 mA, scan range 5 to 30°, scan step size 0.01°, total scan time 33 minutes, VANTEC detector, and antiscattering slit 1 mm. FIGS. 1-7 show characterization data for hydrochloride salt forms.

The hydrochloride salt was polymorphic, adopting crystalline forms exhibiting DSC endotherms at 119° C. (Form III), 127° C. (Form IV), 167° C. (Form II), and 186° C. (Form I). Another form, potentially an ethanol solvate/hydrate, exhibited multiple endotherms, corresponding to 1) desolvation at about 100° C., 2) Form I at about 183° C., and 3) possibly another polymorph at about 200° C. The Table below illustrates certain characteristics of observed hydrochloride salt crystal forms:

Crystal Form Table

| Crystal Form I | | | | |
|---|---|---|---|---|
| Mono- | | | | |
| hydrochloride | | Crystal Form | Crystal Form | |
| (8% HCl) | Crystal Form II | III | IV | Crystal Form V |
| Melting: 180-186° C. | Melting: 165° C. | Melting: 125° C. | Melting: 125° C. | Three peaks: |
| | | | | About 100° C. |
| | | | | About 180° C. |
| | | | | About 200° C. |
| Non-hygroscopic | Somewhat | Hygroscopic | Not tested | Hygroscopic (7% |
| | hygroscopic (5% | (10% water at | | at RH 50%) |
| | water at RH | RH 50%) | | |
| | 50%) | | | |

Of the various observed hydrochloride forms, only Form I (186° C.) is relatively non-hygroscopic, gaining only about 0.5% moisture when equilibrated at RH less than or equal to 70%. At 70-100% RH, Form I gains at least about 2% moisture, but loses it without significant hysteresis on decreasing RH. Evidence of a hydrochloride hydrate was not observed after the hygroscopicity test.

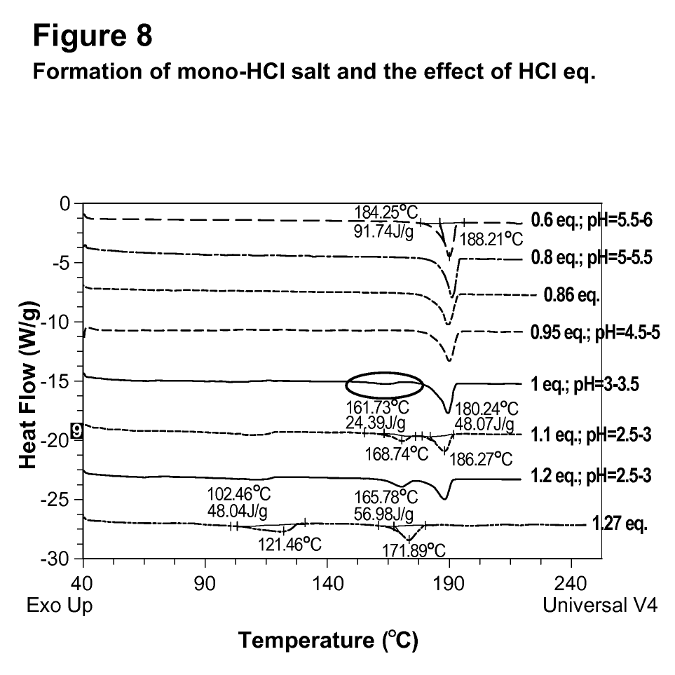

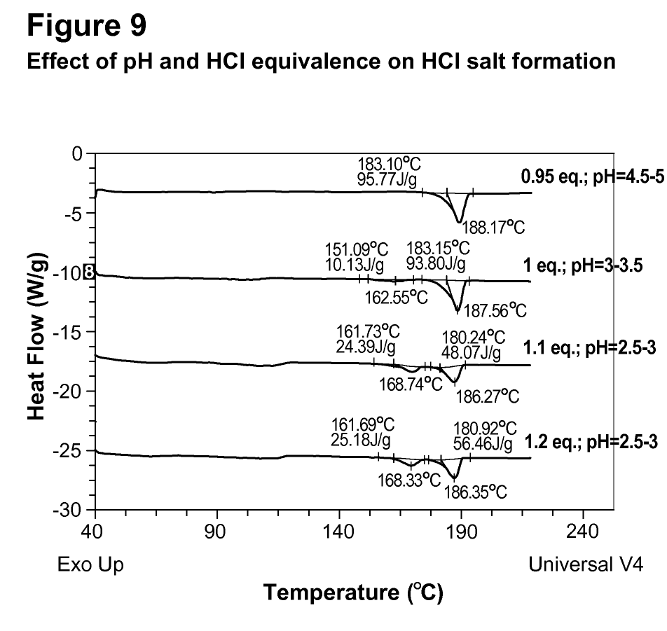

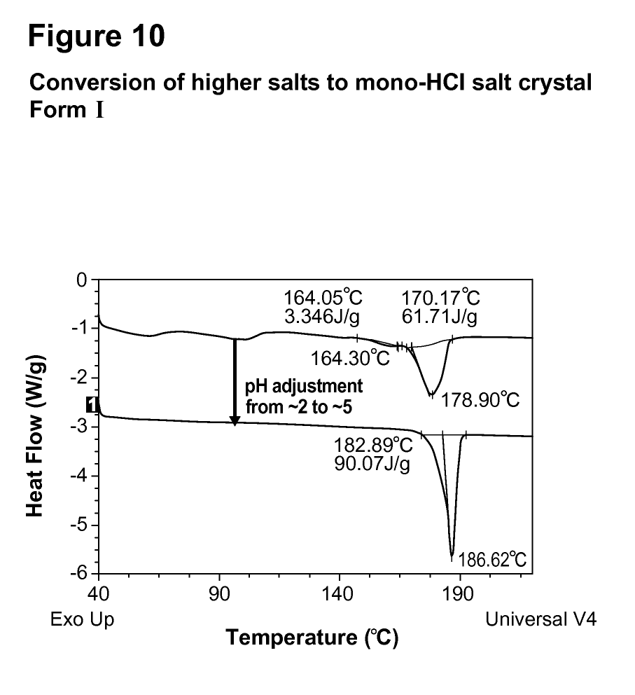

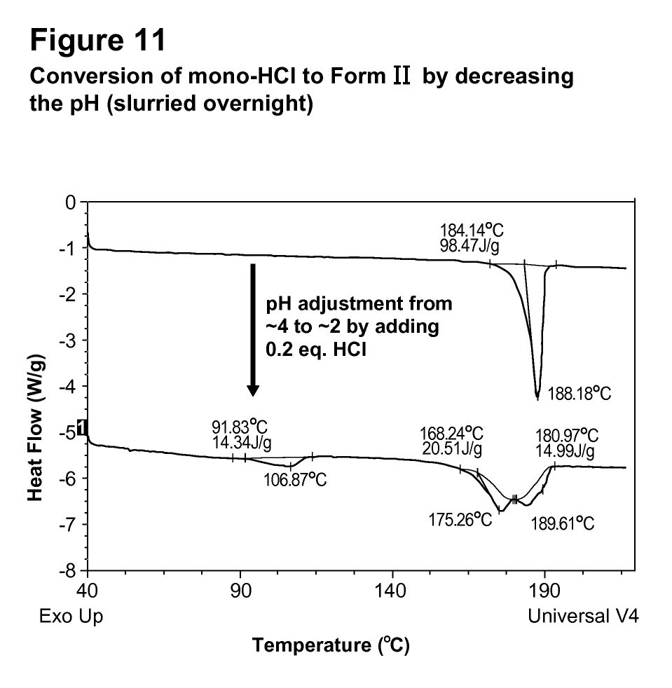

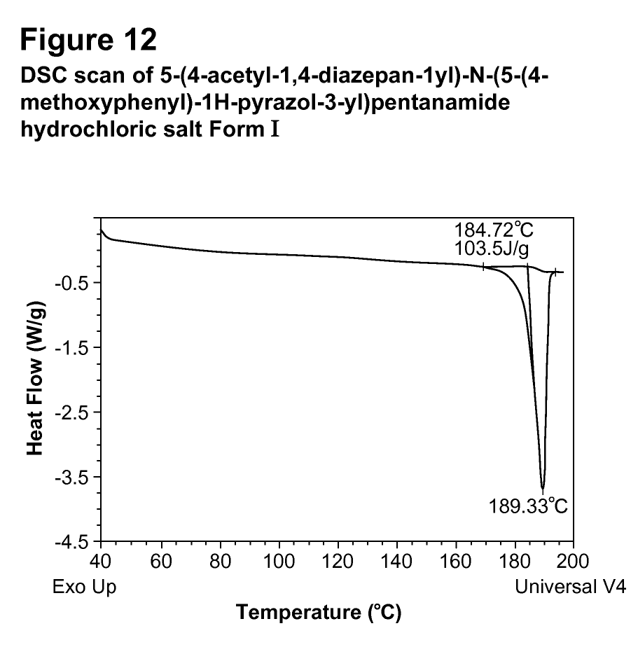

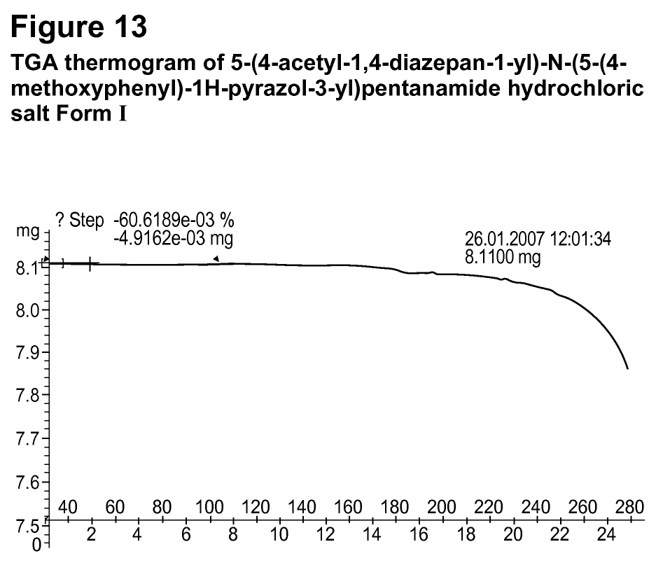

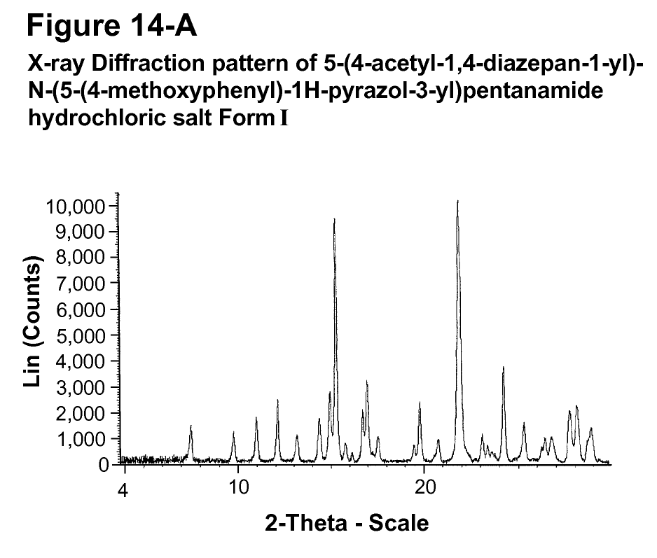

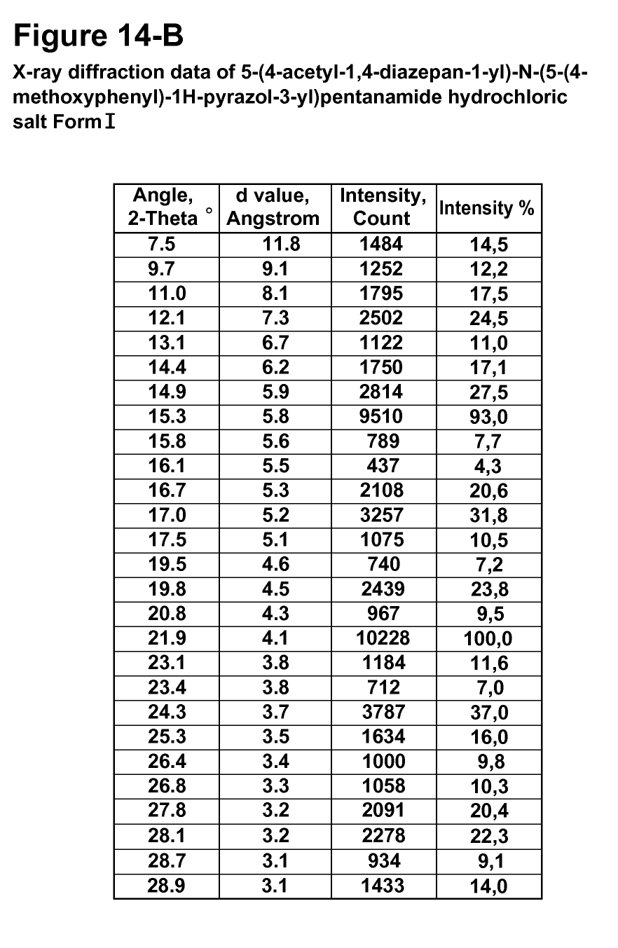

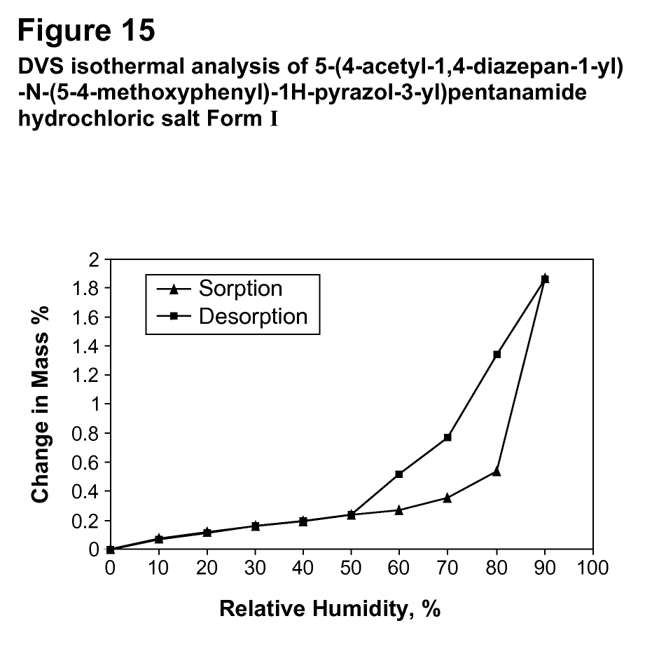

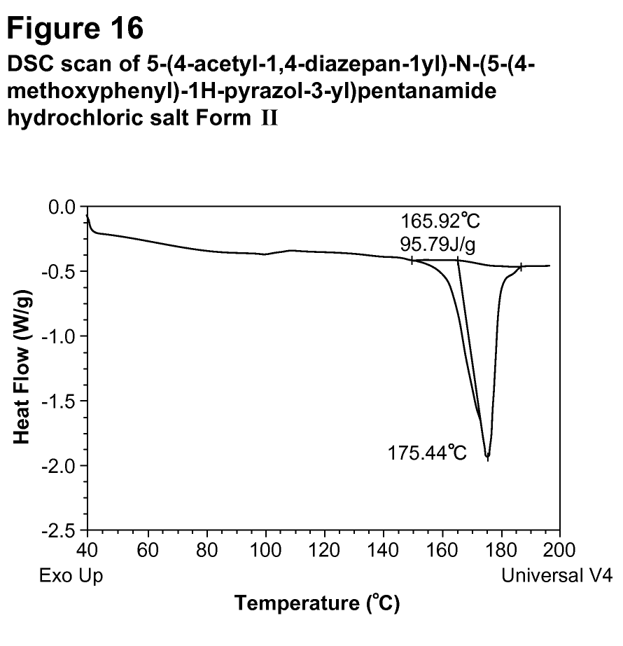

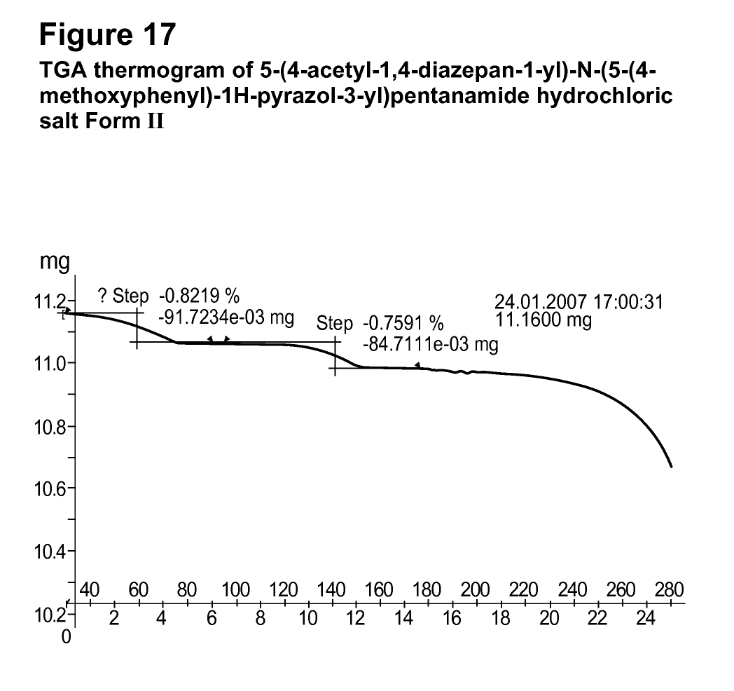

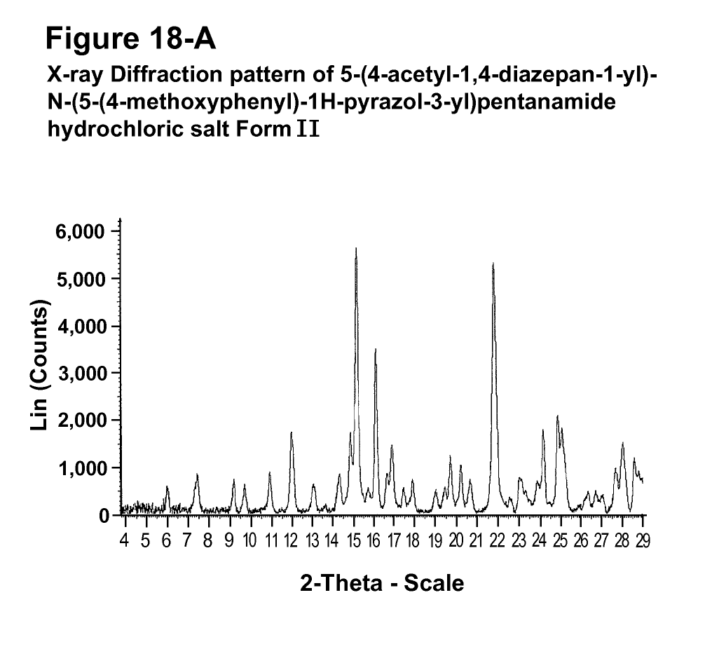

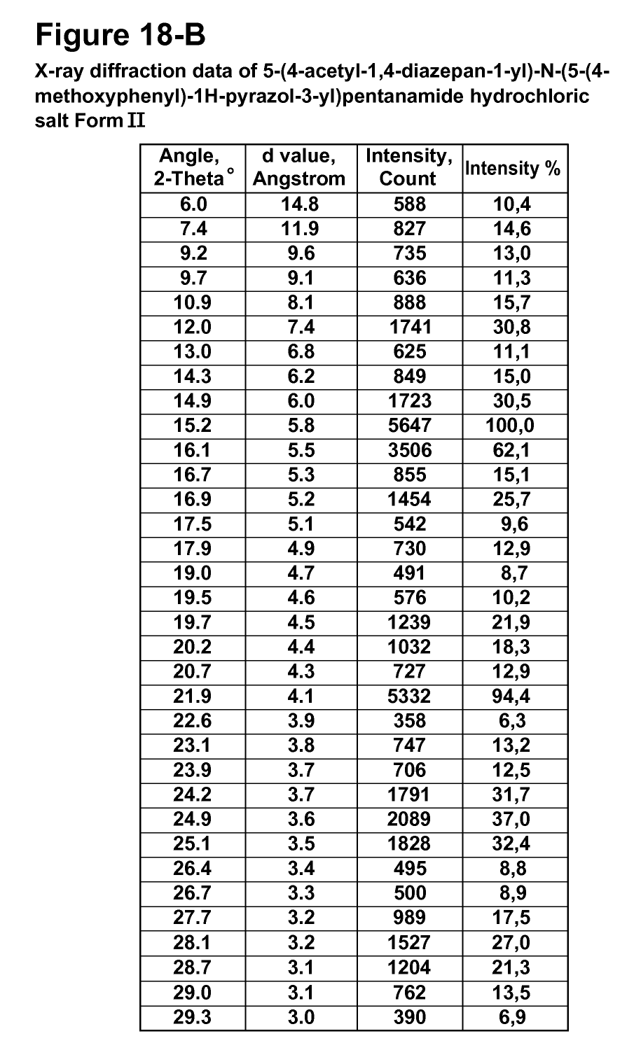

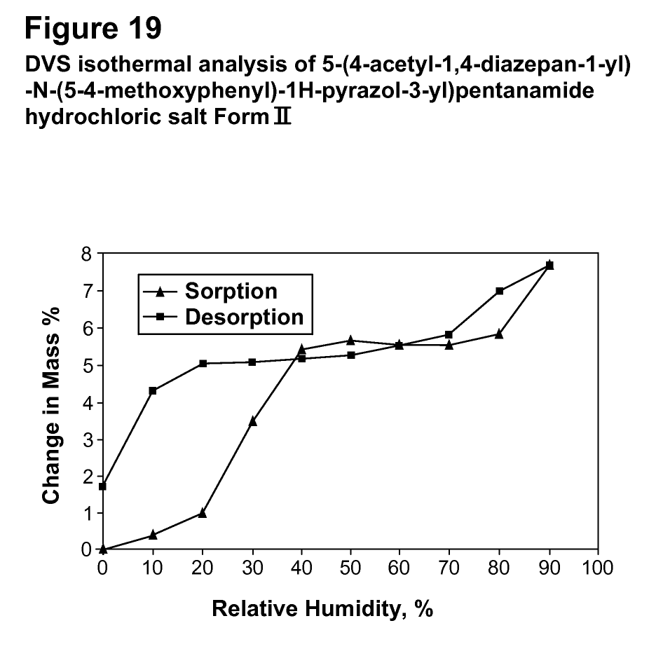

Higher degrees of hydrochloride salt were formed, depending on the amount of hydrochloric acid present in the solution during reactive crystallization. The conversion of higher degrees of hydrochloride salt to mono-hydrochloride salt can be achieved by adjusting the pH of the solution to more than pH 5. Further adjustment, however, can result in formation of inorganic salts. In some embodiments, pure mono-hydrochloride salt forms are produced with hydrochloride equivalence and slurry pH of <0.95 eq. (e.g., 0.93) and pH 5, respectively (see, for example, FIGS. 8-11).

### Example 7

### Characterization of Certain Crystal Forms of 5-(4-Acetyl-[1,4]diazepan-1-yl)-pentanoic acid [5-(4-methoxy-phenyl)-1H-pyrazol-3-yl]-amide hydrochloride Salt

The present Example describes characterization of two surprisingly non-hygroscopic crystal forms (Forms I and II, as described above) of a hydrochloride salt of 5-(4-Acetyl-[1,4]diazepan-1-yl)-pentanoic acid [5-(4-methoxy-phenyl)-2H-pyrazol-3-yl]-amide:

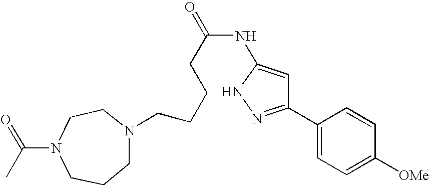

Both forms are considerably soluble in water. The melting point of Form I is 185° C. (plus or minus 2 degrees); the melting point of Form II is 166° C. (plus or minus 5 degrees).

Form I picks up moisture at relative humidity (RH) of about 50% and absorbs up to about 2% water eventually (90% RH) and loses the water as RH decreases (<50%). Form I also exhibits characteristic X-ray peaks at 20 of 15.3° and 21.9°, plus or minus about 0.3°, depending upon the machine and measurement method utilized.

Form II picks up moisture at RH of about 20% and absorbs up to 7% water eventually (RH of 90%) and holds 2% at low RH (0%). Form II also exhibits characteristic X-ray peaks at 2θ of 20.2° and 24.9°, plus or minus about 0.3°, depending upon the machine and measurement method utilized. Differential scanning calorimetry data were collected for each solid form achieved using a DSC (TA instruments, model Q1000) under the following parameters: 50 mL/min purge gas (N2); scan range 40 to 200° C., scan rate 10° C./min.

Thermogravimetric analysis data were collected using a TGA instruments (Mettler Toledo, model TGA/SDTA 851e) under the following parameters: 40 ml/min purge gas (N2); scan range 30 to 250° C., scan rate 10° C./min.

X-ray data were acquired using an X-ray powder diffractometer (Bruker-axs, model D8 advance) having the following parameters: voltage 40 kV, current 40.0 mA, scan range (2θ) 3.7 to 30°, scan step size 0.0°, total scan time 33 minutes, VANTEC detector, and antiscattering slit 1 mm.

Dynamic Vapor Sorption (DVS) was done at 25-26° C.

Results of thermal studies on Crystal Forms I and II are shown in the ensuing Figures.

### Example 8

### Preparation of Crystal Form I of the Hydrochloride Salt of 5-(4-Acetyl-[1,4]diazepan-1-yl)-pentanoic acid [5-(4-methoxy-phenyl)-2H-pyrazol-3-yl]-amide

The present Example describes the preparation of crystal form I of the hydrochloride salt of 5-(4-Acetyl-[1,4]diazepan-1-yl)-pentanoic acid [5-(4-methoxy-phenyl)-2H-pyrazol-3-yl]-amide.

First procedure: 611.7 mg of the free base form of 5-(4-Acetyl-[1,4]diazepan-1-yl)-pentanoic acid [5-(4-methoxy-phenyl)-2H-pyrazol-3-yl]-amide was dissolved in 1.97 mL acetone at 35° C. A solution of 5% HCl in acetone-water was prepared by diluting 37.5% aq. HCl using acetone. 0.6 ml of 5% HCl was added slowly. 1.2 ml EtOH ASDQ (100:10 ethanol:methanol) was added slowly. The solution became milky in a few minutes; stirring was performed for around 5 minutes. 0.25 ml of 5% HCl was added slowly. After 5 minutes, 0.25 ml of 5% HCl was added slowly. After 5 minutes, 0.087 ml of 5% HCl was added slowly. The mixture was heated to about 40-50° C. The mixture was left at room temperature while stirring overnight. Crystals were filtered and washed with 2 ml acetone, and were dried at 45° C. for about 7 hours. 505 mg of solid were recovered.

Second procedure: 377 mg of the free base form of 5-(4-Acetyl-[1,4]diazepan-1-yl)-pentanoic acid [5-(4-methoxy-phenyl)-2H-pyrazol-3-yl]-amide was dissolved in 1.2 ml acetone at 35° C. 0.754 ml ethanol ASDQ (100:10 ethanol:methanol) was added. A solution of 5% HCl in acetone-water was prepared by diluting 37.5% aq HCl using acetone. 0.18 ml diluted HCl solution was added slowly. A seed of crystal form I of the hydrochloride salt of 5-(4-Acetyl-[1,4]diazepan-1-yl)-pentanoic acid [5-(4-methoxy-phenyl)-2H-pyrazol-3-yl]-amide was added. 0.18 ml diluted HCl solution was added slowly. Around two minutes later, 0.18 ml diluted HCl solution was added slowly. Around two minutes later, another 0.18 ml diluted HCl solution was added slowly. The mixture was heated to about 40-50° C., and then was left at room temperature while stirring overnight. The crystals were filtered and washed with 1.5 ml acetone, and were dried at 45° C. for about 6 hours.

### Example 9

### Alternative purification of the hydrochloride salt of 5-(4-Acetyl-[1,4]diazepan-1-yl)-pentanoic acid [5-(4-methoxy-phenyl)-2H-pyrazol-3-yl]-amide

The hydrochloride salt of 5-(4-Acetyl-[1,4]diazepan-1-yl)-pentanoic acid [5-(4-methoxy-phenyl)-2H-pyrazol-3-yl]-amide (compound I) may be re-purified by basifying the hydrochloride salt and extracting the free base into a suitable solvent (e.g. methylene chloride). The organic extracts may be washed with water. The organic phase is concentrated and the solvent switched to ethanol. Acetone is added to give a solution of compound A which was clarified and mixed with ethanol, acetone, hydrochloric acid, and water. Acetone is added, the solids filtered, washed with mixture of acetone, water and dried to give compound I. A representative procedure is described below.

To a suitable reactor, compound I (0.2 kg) was dissolved in water (0.80 L) and clarified through a filter pad. To the filtrates was added methylene chloride (2.65 kg) and cooled to 15° C. A 30% aqueous solution of sodium hydroxide (0.062 kg) was added over 30 mins and mixed for 20 mins. The pH was >8. The layers were separated; the organic layer was washed with water (2×0.40 kg) and distilled down to 0.46 L forming a hazy mixture. The methylene chloride solvent was exchanged with ethanol by vacuum distillation chases (2×0.79 kg).

Acetone (0.63 kg) was added to the concentrate and the solution clarified. An accurate strength of the free base was determined of the concentrate. Water (0.065 kg) was added to form a clear solution. A solution of 5% HCl (0.043 kg) in acetone (0.14 kg) and alcohol (0.14 kg of ethanol:acetone (91:9) v/v) was prepared and stirred until homogeneous at 10° C. About one third of the 5% HCl solution (0.098 kg) was added to the reactor over a minimum of 20 min., maintaining the temperature in the range of 20-25° C. A second third of the 5% HCl solution (0.098 kg) was then added to the reactor over a minimum of 20 min., maintaining the temperature in the range of 20-25° C. The contents of the reactor were seeded with 75 mg of 5-(4-acetyl-[1,4]diazepan-1-yl)-N-[5-(4-methoxy-phenyl)-1H-pyrazol-3-yl]-pentanamide HCl (e.g., Form 1), followed by the addition of the last third of 5% HCl solution (0.098 kg) over a minimum of 20 min., maintaining the temperature in the range of 20-25° C. The contents of the reactor were seeded with another 75 mg of 5-(4-acetyl-[1,4]diazepan-1-yl)-N-[5-(4-methoxy-phenyl)-1H-pyrazol-3-yl]-pentanamide HCl (e.g., Form 1). Another 0.08 equiv. of the 5% HCl solution (0.029 kg) was then added to the reactor over a minimum of 30 min., maintaining the temperature in the range of 20-25° C. Judicious monitoring of pH was performed to attain the desired pH range of 5.2-5.8. The mixture was stirred at 20-25° C. for a minimum of 1 hr., forming a thin suspension. Acetone (0.63 kg) was added over a minimum of 60 min., maintaining the temperature in the range of 20-25° C. The mixture was stirred at 20-25° C. for a minimum of 60 min. Acetone (1.58 kg) was added to the reactor over a minimum of 3 hr., maintaining the temperature in the range of 20-25° C., forming a thick suspension. The mixture was then stirred at 20-25° C. for a minimum of 12 h. Crystallization was considered complete when there was <15% of the product present in the mother liquor. Longer stirring was employed if crystallization was not complete. The mixture was then filtered on a Buchner funnel (polypropylene pad) using house vacuum. A solution of water (0.012 kg), acetone (0.24 kg) and 0.063 kg alcohol (ethanol:acetone (91:9) v/v) was stirred until homogeneous (20% ethanol, 3% water, 77% acetone overall). This solution was used to wash the filter cake. A solution of water (0.012 kg), acetone (0.18 kg) and 0.13 kg alcohol (ethanol:acetone (91:9) v/v) was stirred until homogeneous (40% ethanol, 3% water, 57% acetone overall). This solution was used to wash the filter cake. The wet cake was subjected to suction under nitrogen using house vacuum and held for 30 min. after dripping stopped. Product purity was checked by HPLC and additional washing was performed if total impurities were not ≦2%. Product was oven dried in a vacuum oven with nitrogen bleed at 38-45° C., maintaining vacuum at 20 torr for a minimum of 12 h until loss on drying of less than 1% was obtained. Following drying, 0.17 kg of the title compound was obtained in 85% yield.

We claim:

1. A method comprising the steps of:
providing a compound of formula I-1: 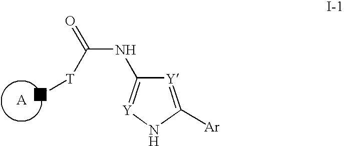
wherein:
Ring A is a 4-8 membered saturated ring, having 0-2 heteroatoms independently selected from O, N, or S in addition to the nitrogen depicted in Ring A, wherein Ring A is independently substituted with 0-4 R′ groups;
R′ is selected from the group consisting of mono- or di-[linear, branched or cyclic C₁₋₆ alkyl]aminocarbonyl; linear, branched or cyclic C₁₋₆ alkyl, alkoxy, or acyl;
Y and Y′ are each independently N or C, with the proviso that at least one of Y or Y′ is N;
T is a C₃₋₅ bivalent hydrocarbon chain, optionally carrying an oxo group and optionally substituted with one or more halogen, hydroxy, C₁₋₅ alkyl, alkoxy, fluoroalkyl, hydroxyalkyl, alkylidene, or fluoroalkylidene groups; C₃₋₆ cycloalkane-1,1-diyl, oxacycloalkane-1,1-diyl, C₃₋₆ cycloalkane-1,2-diyl, or oxacycloalkane-1,2-diyl groups, wherein the bonds of the 1,2-diyl radical form a fused ring with the T chain; and with the proviso that when T carries an oxo group, said oxo group is not part of an amide bond; and
Ar is a group selected from 6-10 membered aryl, or 5-10 membered heteroaryl having 1-4 heteroatoms independently selected from nitrogen, oxygen or sulfur; wherein Ar is optionally substituted with one or more substituents independently selected from halogen; hydroxy; mercapto; cyano; nitro; amino; sulfonyl; linear, branched or cyclic (C1-C6) alkyl, trihaloalkyl, di- or trihaloalkoxy, alkoxy, or alkylcarbonyl; (C3-C6) cycloalkyl-(C1-C6) alkoxy; (C3-C6) cycloalkyl-(C1-C6) alkyl; linear, branched, or cyclic (C1-C6) alkylcarbonylamino; mono- or di-, linear, branched, or cyclic (C1-C6) alkylaminocarbonyl; carbamoyl; linear, branched, or cyclic (C1-C6) alkylsulphonylamino; linear, branched, or cyclic (C1-C6) alkylsulphonyl; mono- or di-, linear, branched, or cyclic (C1-C6) alkylsulphamoyl; linear, branched or cyclic (C1-C6) alkoxy-(C1-C6) alkyl; wherein, two substituents may be taken together with their intervening atoms to form a ring; and
(b) treating said compound of formula I-1 with hydrochloric acid in a ternary solvent solvent system to form a compound of formula I-1′:

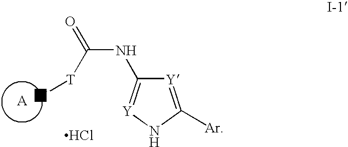

2. The method according to claim 1, wherein the ternary solvent system comprises acetone, water, and ethanol.

3. The method according to claim 2, wherein the hydrochloric acid is added as about a 5% solution in acetone and ethanol to a compound of formula I-1 in a mixture of acetone and water.

4. The method according to claim 3, wherein about 0.93 equivalents of hydrochloric acid is added relative to a compound of formula I-1.

5. The method according to claim 1, further comprising the steps of:
(c) providing compound of formula B′: 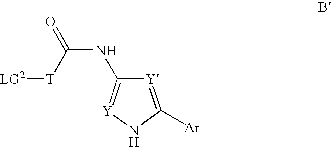
wherein, LG² is a suitable leaving group, and
(d) treating said compound of formula B′ with a compound of formula G′: 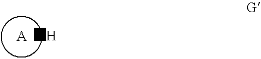
optionally in the presence of a suitable base and/or additive, to form compound I-1.

6. The method according to claim 5, wherein LG² is selected from Br, I, OMs, OTs, or OTf.

7. The method according to claim 6, wherein LG² is Br.

8. The method according to claim 5, further comprising the steps of:
(e) providing a compound of formula C′: 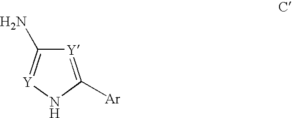
and
(f) treating said compound of formula C′ in the presence of a suitable base with a compound of formula F′: 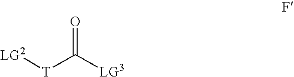
wherein LG is a suitable leaving group, to form a compound of formula B′.

9. The method of claim 1, wherein the compound of formula I-1 is compound A:

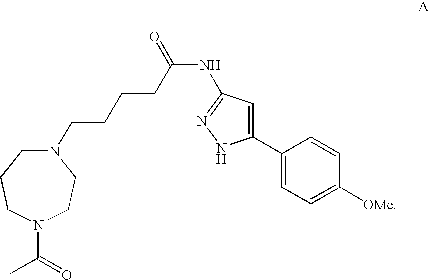

10. The method of claim 1, wherein the compound of formula I-1′ is compound I:

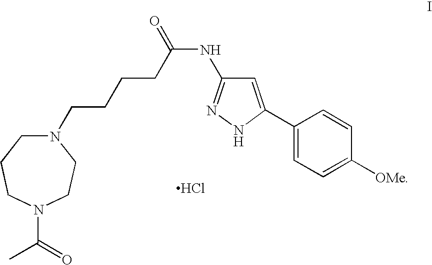

11. The method of claim 9, wherein the hydrochloric acid is added as about a 5% solution in acetone and ethanol to a compound of formula A in a mixture of acetone and water.

12. The method according to claim 11, wherein about 0.93 equivalents of hydrochloric acid is added relative to a compound A.

13. The method of claim 5, wherein the compound of formula B′ is:

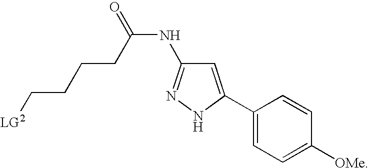

14. The method according to claim 13, wherein LG² is selected from Br, I, OMs, OTs, or OTf.

15. The method according to claim 14, wherein LG² is Br.

16. The method according to claim 13, wherein the base is pyridine, diisopropylethylamine, triethylamine, sodium bicarbonate, sodium carbonate, potassium carbonate, or combinations thereof.

17. The method according to claim 16, wherein the base is diisopropylethylamine.

18. The method according to claim 16, wherein the base is potassium carbonate.

19. The method according to claim 13, wherein the additive is an iodide source selected from sodium iodide, potassium iodide, hydrogen iodide, tetralkylammonium iodide, or a mixture thereof.

20. The method according to claim 19, wherein the iodide source is potassium iodide.

21. The method according to claim 19, wherein the iodide source is sodium iodide.

22. The method of claim 8, wherein the compound of formula C′ is: 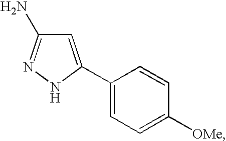
and the compound of formula F′ is:

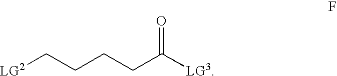

23. The method according to claim 22, wherein LG³ is selected from halogen, OR, 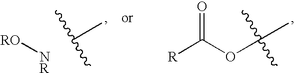
wherein each R is independently hydrogen or an optionally substituted group selected from C₁₋₆ aliphatic, 6-10 membered aryl, or 5-10 membered heteroaryl having 1-4 heteroatoms independently selected from nitrogen, oxygen or sulfur.

24. The method according to claim 23, wherein LG³ is Cl.

25. The method according to claim 22, wherein LG² is halogen.

26. The method according to claim 22, wherein a compound of formula F is selected from 5-bromovaleryl chloride or 5-iodovaleryl chloride.

27. The method according to claim 26, wherein a compound of formula F is 5-bromovaleryl chloride.

28. The method of claim 22, further comprising the steps of:
(g) providing compound D: 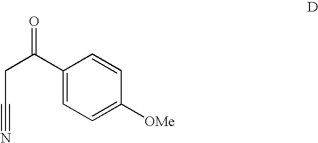
and
(h) treating said compound D with hydrazine, or an equivalent thereof, to form compound
C:

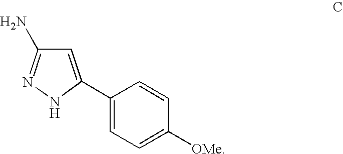

29. The method of claim 28, further comprising the steps of:
(a) providing a compound of formula E: 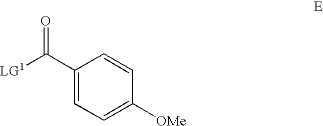
wherein, LG¹ is a leaving group, and
(b) treating said compound of formula with acetonitrile to form a mixture thereof, and
(c) treating said mixture with a suitable base to give compound D:

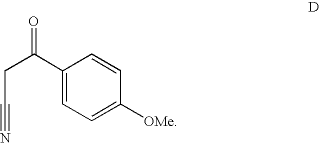

30. The method according to claim 29, wherein LG¹ is a halogen, alkoxy, sulphonyloxy, optionally substituted alkylsulphonyl, optionally substituted alkenylsulfonyl, optionally substituted arylsulfonyl, or diazonium moiety.

31. The method according to claim 30, wherein LG¹ is methoxy.

32. A method comprising the steps of:
(a) providing a compound of formula C′: 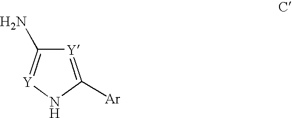
and
(b) treating said compound of formula C′ in the presence of a suitable base with a compound of formula F′: 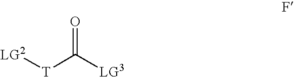
wherein LG² and LG³ are suitable leaving groups, to form a compound of formula B′: 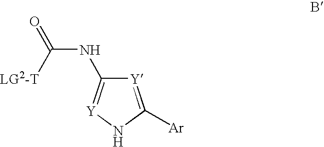
and
(c) treating said compound of formula B′ with a compound of formula G′: 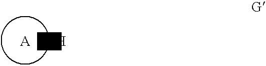
optionally in the presence of a suitable base and/or additive, to form compound I-1: 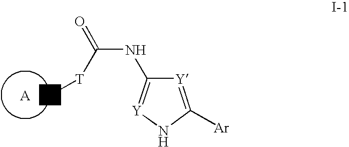
and
(d) treating said compound of formula I-1 with hydrochloric acid in a ternary solvent solvent system to form a compound of formula I-1′: 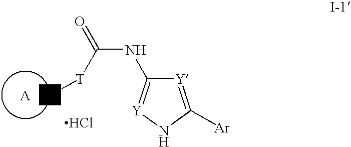
wherein Ring A is a 4-8 membered saturated ring, having 0-2 heteroatoms independently selected from O, N, or S in addition to the nitrogen depicted in Ring A, wherein Ring A is independently substituted with 0-4 R′ groups;
R′ is selected from the group consisting of mono- or di-[linear, branched or cyclic C₁₋₆ alkyl]aminocarbonyl; linear, branched or cyclic C₁₋₆ alkyl, alkoxy, or acyl;
Y and Y′ are each independently N or C, with the proviso that at least one of Y or Y′ is N;
T is a C₃₋₅ bivalent hydrocarbon chain, optionally carrying an oxo group and optionally substituted with one or more halogen, hydroxy, C₁₋₅ alkyl, alkoxy, fluoroalkyl, hydroxyalkyl, alkylidene, or fluoroalkylidene groups; C₃₋₆ cycloalkane-1,1-diyl, oxacycloalkane-1,1-diyl, C₃₋₆ cycloalkane-1,2-diyl, or oxacycloalkane-1,2-diyl groups, wherein the bonds of the 1,2-diyl radical form a fused ring with the T chain; and with the proviso that when T carries an oxo group, said oxo group is not part of an amide bond; and
Ar is a group selected from 6-10 membered aryl, or 5-10 membered heteroaryl having 1-4 heteroatoms independently selected from nitrogen, oxygen or sulfur; wherein Ar is optionally substituted with one or more substituents independently selected from halogen; hydroxy; mercapto; cyano; nitro; amino; sulfonyl; linear, branched or cyclic (C1-C6) alkyl, trihaloalkyl, di- or trihaloalkoxy, alkoxy, or alkylcarbonyl; (C3-C6) cycloalkyl-(C1-C6) alkoxy; (C3-C6) cycloalkyl-(C1-C6) alkyl; linear, branched, or cyclic (C1-C6) alkylcarbonylamino; mono- or di-, linear, branched, or cyclic (C1-C6) alkylaminocarbonyl; carbamoyl; linear, branched, or cyclic (C1-C6) alkylsulphonylamino; linear, branched, or cyclic (C1-C6) alkylsulphonyl; mono- or di-, linear, branched, or cyclic (C1-C6) alkylsulphamoyl; linear, branched or cyclic (C1-C6) alkoxy-(C1-C6) alkyl; wherein, two substituents may be taken together with their intervening atoms to form a ring;
wherein each step is performed sequentially without isolation of intermediates B′ or I-1.

33. The method according to claim 32, wherein the compound of formula F′ is slowly added to the compound of formula C′ in step (b).

34. The method according to claim 33, wherein about 0.5-0.95 equivalents of hydrochloric acid is added relative a compound of formula I-1.
